(19) United States
(12) Patent Application Publication
(10) Pub. No.: US 2026/0224591 A1
(43) Pub. Date: Aug. 6, 2026

(54) USE OF A FIXED COMBINATION OF HALOBETASOL PROPIONATE AND TAZAROTENE TO TREAT CONDITIONS MEDIATED BY INTERLEUKIN-17A

(71) Applicant: Bausch Health Ireland Limited, Dublin (IE)

(72) Inventors: Abby A. JACOBSON, Bridgewater, NJ (US); Tina LIN, Irvine, CA (US)

(21) Appl. No.: 19/142,832

(22) PCT Filed: Dec. 29, 2023
(86) PCT No.: PCT/EP2023/087982

§ 371 Date: Jun. 24, 2025

Related U.S. Application Data
(60) Provisional application No. 63/436,583, filed on Dec. 31, 2022.

(51) Int. Cl. A61K 31/573; A61K 9/00; A61K 31/192; A61K 31/4436; A61K 31/58; A61P 17/06; A61P 29/00

(52) U.S. Cl. CPC A61K 31/573; A61K 9/0014; A61K 31/192; A61K 31/4436; A61K 31/58; A61P 17/06; A61P 29/00

(57) ABSTRACT

The invention relates generally to compositions and their therapeutic use. More particularly, the invention relates to compositions that modulate the activity of IL-17 and/or are useful in the treatment of skin diseases and conditions, such as inflammatory, autoimmune diseases, or oncological diseases and conditions, and other IL-17-associated disorders.

### CROSS-REFERENCES TO RELATED APPLICATIONS

This application claims the benefit of U.S. Provisional Application No. 63/436,583 filed Dec. 31, 2022, which is incorporated by reference herein in its entirety.

### BACKGROUND OF THE INVENTION

IL-17 is a cytokine whose family consists of six members, from IL-17A to IL-17F, even if the term IL-17 usually refers to IL-17A (see, Amatya N, et al. IL-17 signaling: the yin and the yang. Trends Immunol 2017; 38:310-322). All the family members share some activities: IL-17A, IL-17F, and their heterodimers IL-17A/F have inflammatory effect with different strength; IL-17B, IL-17C, IL-17D are proinflammatory cytokines with unknown role. The IL-17E cytokine, also named IL-25, is involved in Th2 cells response (see, Gaffen S L. Structure and signalling in the IL-17 receptor family. Nat Rev Immunol 2009; 9:556-567).

Production of IL-17 is mainly operated by T helper type 17 (Th17), CD8⁺ T cells, γδ T cells, invariant natural killer T cells (INKT), natural killer (NK) cells, natural Th17 cells, and lymphoid tissue inducer (LTi) cells (see, Amatya N, et al. IL-17 signaling: the yin and the yang. Trends Immunol 2017; 38:310-322).

IL-17 exerts many physiological functions including: neutrophil recruitment, Th2 stimulation to provide an effective response against extracellular organisms, macrophage production of IL-1β and TNF-α, and inflammatory mediator matrix metalloproteinases (MMPs) induction (see, Amatya N, et al. IL-17 signaling: the yin and the yang. Trends Immunol 2017; 38:310-322).

Despite of the important role of IL-17 cytokine in regulating adaptive and innate immune systems, its overproduction could be involved in several diseases (see, Sharma J, et al. A knowledgebase resource for interleukin-17 family mediated signaling. J Cell Commun Signal 2015; 9:291-196; and Beringer A, et al. IL-17 in chronic inflammation: from discovery to targeting. Trends Mol Med 2016; 22:230-241).

In recent years, different studies have tried to associate the IL-17 pathway to an increasing number of inflammatory diseases, such as asthma, chronic obstructive pulmonary disease (COPD), lupus, polymyalgia rheumatica, giant cell arteritis, Behçet disease, dry-eye syndrome, Sjögren's syndrome, Crohn's disease, and multiple sclerosis. However, the role of IL-17 is still unclear (see, Beringer A, et al. IL-17 in chronic inflammation: from discovery to targeting. Trends Mol Med 2016; 22:230-241). Th17 cells and IL-17 have also been identified to be implicated in the pathogenesis of several human autoimmune diseases, such as rheumatoid arthritis, multiple sclerosis, inflammatory bowel disease, systemic sclerosis, primary Sjogren's syndrome, alopecia areata, and vitiligo (see, Maddur M S, et al. Th17 cells: biology, pathogenesis of autoimmune and inflammatory diseases, and therapeutic strategies. Am J Pathol 2012; 576:8-18). Furthermore, a recent study reported a correlation between the serum levels of IL-17 and activity of the disease in selected autoimmune bullous skin diseases, such as dermatitis herpetiformis (DH), bullous pemphigoid (BP), and pemphigus vulgaris (PV) (see, Maddur M S, et al. Th17 cells: biology, pathogenesis of autoimmune and inflammatory diseases, and therapeutic strategies. Am J Pathol 2012; 576:8-18).

It can be seen from above that modulation of IL-17 has important therapeutic implications. Although various antibodies to IL-17 have been described in the scientific literature, very few small molecule-type modulators of IL-17 are known. Accordingly, there is a need for the development of small moleucle-type modulators of IL-17 and compositions thereof that can be used topically to treat skin or cutaneous diseases or conditions associate with elevated levels of IL-17.

### BRIEF SUMMARY OF THE INVENTION

The invention disclosed herein provides compositions, methods for modulating the activity of IL-17, and methods for treating various skin conditions and diseases using such compositions.

In one aspect, the invention provides topical pharmaceutical compositions comprising a combination of: (a) a corticosteroid or a pharmaceutically acceptable salt or ester thereof; and (b) a retinoid or a pharmaceutically acceptable salt or ester thereof, and methods using the same, for modulating IL-17 activity and/or for treating skin diseases and conditions associate with elevated IL-17 activity. In one embodiment, the disease or condition associated with elevated IL-17 activity is an inflammatory, an autoimmune, or an oncological disease, condition, or disorder.

In another aspect, the invention relates to topical pharmaceutical compositions comprising a combination of: (a) halobetasol or a pharmaceutically acceptable salt or ester thereof; and (b) tazarotene or a pharmaceutically acceptable tazarotenic acid salt, and methods using the same, for modulating IL-17 activity and/or for treating skin diseases and conditions associate with elevated IL-17 activity. In one embodiment, the disease or condition associated with elevated IL-17 activity is an inflammatory, an autoimmune, or an oncological disease, condition, or disorder.

In yet another aspect, the invention provides topical pharmaceutical compositions comprising a combination of: (a) halobetasol or a pharmaceutically acceptable salt or ester thereof; and (b) a tazarotene or a pharmaceutically acceptable tazarotenic acid salt or a pharmaceutically acceptable non-ethyl ester of tazarotenic acid, and methods using the same, for modulating IL-17 activity and/or for treating skin diseases and conditions associate with elevated IL-17 activity; wherein each of: (a) halobetasol or a pharmaceutically acceptable salt or ester thereof; and (b) tazarotene or a pharmaceutically acceptable tazarotenic acid salt or a pharmaceutically acceptable non-ethyl ester of tazarotenic acid is present in the composition at a positive concentration of less than 0.09% based on the weight of the composition (e.g., each active is present in the composition at a concentration from about 0.001% to about 0.9% based on the weight of the composition). A non-ethyl ester of tazarotenic acid is an ester of tazarotenic acid that is not the ethyl ester. For example, a non-ethyl ester can be methyl, propyl, isopropyl, butyl, isobutyl, pentyl, isopentyl, aryl, phenyl, or naphthyl ester. In one embodiment, the concentration of halobetasol or a pharmaceutically acceptable salt is in the range from about 0.001 to about 0.049 weight percent (“wt %”); and the concentration of tazarotene or a pharmaceutically acceptable tazarotenic acid salt is in the range from about 0.001 to about 0.049 wt %.

In still another aspect, the invention provides a method of treating a skin disease or condition associated with elevated levels of IL-17 in a subject in need thereof, the method comprising applying to an affected area on the body of the subject a composition comprising: (a) a corticosteroid or a pharmaceutically acceptable salt or ester thereof, at a positive concentration of less than 0.05 percent by weight of the composition; (b) a retinoid or a pharmaceutically acceptable salt or ester thereof at a positive concentration of less than 0.09 percent by weight of the composition; and (c) a dermatologically acceptable excipient. In one embodiment, the corticosteroid includes, but is not limited to, halobetasol, clobetasol, betamethasone, diflorasone, and fluocinonide. In one embodiment, the retinoid is tazarotene.

In another aspect, the invention provides a method of treating a patient suffering from a skin disease or condition associated with elevated levels of IL-17, the method comprising: (a) determining whether the patient has an elevated level of IL-17; and (b) if the patient has an elevated level of IL-17, administering to the patient an effective amount of a composition comprising or susceptible to a medical condition that is mediated directly or indirectly by IL-17, the method comprising administering to the patient a composition for a time sufficient to treat the disease or condition, wherein the composition comprises: (a) a corticosteroid or a pharmaceutically acceptable salt or ester thereof, at a positive concentration of less than 0.05 percent by weight of the composition; (b) a retinoid or a pharmaceutically acceptable salt or ester thereof at a positive concentration of less than 0.09 percent by weight of the composition; and (c) a dermatologically acceptable excipient. In one embodiment, the corticosteroid includes, but is not limited to, halobetasol, clobetasol, betamethasone, diflorasone, and fluocinonide. In one embodiment, the retinoid is tazarotene.

In still another aspect, the invention provides a method of treating a subject suffering from a disease or condition associate with elevated levels of IL-17 comprising the steps of: (a) determining whether the subject has an elevated level of one or more IL-17 induced chemokine or effector; and (b) if the subject does have an elevated level of one or more IL-17 chemoline or effector, topically applying to an affected area of the body of the subject a composition of the invention. In one embodiment, the chemokine or effector is one or more of IL-6, IL-8, G-CSF, TNF-α, PGE2, and INF-γ.

Other features and advantages of the invention will become apparent from the accompanying drawings and the following detailed description and claims.

### BRIEF DESCRIPTION OF FIGURES

The presently disclosed embodiments will be further explained with reference to the attached drawings. The drawings shown are not necessarily to scale, with emphasis instead generally being placed upon illustrating the principles of the presently disclosed embodiments.

FIG. 1 is a graph illustrating improvement in the visual investigator and subject psoriasis assessments from the TNF-α ELISA findings.

FIG. 2 is a graph illustrating the calibration curve used in the TNF-α ELISA Assessment.

FIG. 3 is a graph illustrating the IL-17A level reduction in the psoriatic plaque from the IL-17A ELISA Assessment.

FIG. 4 is a graph illustrating the calibration curve used in the IL-17A ELISA Assessment.

### DETAILED DESCRIPTION OF THE INVENTION

In general, the invention provides topical compositions and methods for treating skin or cutaneous diseases and conditions associated with elevated levels of IL-17. It has been unexpectedly discovered that topical dermatological compositions comprising two different active ingredients selected from the group consisting of dermatological immune response-modulating agents, such as a corticosteroid, and anti-proliferative agents, such as a retinoid, can modulate, i.e., inhibit, IL-17 activity. It has also been unexpectedly discovered that such topical dermatological compositions are synergistically more efficacious than the combined efficacies of compositions, each comprising only one of the active ingredients in modulating IL-17 activity. In one aspect, one active ingredient is a corticosteroid, such as halobetasol propionate, and the other active ingredient is a retinoid, such as tazarotene.

A. Compositions for Treating Diseases and Conditions Associated with Elevated Levels of IL-17

Throughout this disclosure, unless otherwise indicated, the concentration of an ingredient of the compositions disclosed herein is in percent by weight of the total composition.

In one aspect, the invention provides topical pharmaceutical compositions comprising a combination of: (a) a corticosteroid or a pharmaceutically acceptable salt or ester thereof; and (b) a retinoid or a pharmaceutically acceptable salt or ester thereof, and methods using the same, for treating a disease or condition associated with elevate levels of IL-17 or elevated levels of an IL-17 induced chemokine or effector (such as IL-6, IL-8, G-CSF, TNF-α, PGE2, and INF-γ).

In one aspect, a corticosteroid included in a composition of the invention is selected from the corticosteroids of class 1. The classification of corticosteroids and examples thereof are disclosed in Table 1.

TABLE 1
Class 1 - Superpotent Topical Corticosteroids

Clobetasol propionate
Betamethasone dipropionate
Halobetasol propionate
Fluocinonide
Diflorasone diacetate

Clobetasol propionate and halobetasol propionate have been used for treating steroid responsive dermatoses, such as psoriasis, typically at a concentration of 0.05%, in various dosage forms including ointments, creams, solutions, sprays, and gels.

Betamethasone dipropionate has been used for treating steroid responsive dermatoses, such as psoriasis, typically at a concentration of 0.064% equivalent to 0.05% betamethasone, in several dosage forms including ointments, creams, gels and lotions.

Fluocinonide has been used for treating steroid responsive dermatoses, such as psoriasis, at various concentrations ranging from 0.001% to 0.1%, in various dosage forms including ointments, creams, gels and solutions. Only the 0.1% formulations of fluocinonide have been classified as superpotent.

Diflorasone diacetate has been used for treating steroid responsive dermatoses, such as psoriasis, at various concentration of 0.05%, as an ointment and as a cream. Only ointment formulation has been classified as superpotent.

HPA axis suppression from long term use topical preparations containing clobetasol propionate and halobetasol propionate and other super potent corticosteroids has limited the duration of therapy allowed by FDA. The duration on therapy allowed in the labeling for super potent topical corticosteroids is typically limited to two weeks.

In one aspect, the corticosteroid is present at a concentration below that which is presently utilized in most topical formulations. The formulation contains the corticosteroid at a positive concentration less than 0.05% w/w.

In certain embodiments of the invention, a corticosteroid or a pharmaceutically acceptable salt or ester thereof is present in the composition at a positive concentration of less than 0.05% based on the weight of the composition. Alternatively, the corticosteroid is present in the composition at a concentration in the range from about 0.001 to about 0.049 wt %, or from about 0.005 to about 0.04 wt %, or from about 0.005 to about 0.035 wt %, or from about 0.005 to about 0.03 wt %, or from about 0.005 to about 0.025 wt %, or from about 0.005 to about 0.015 wt %, or from about 0.005 to about 0.01 wt %.

In still another aspect, a retinoid included in a composition of the invention is selected from the group consisting of tazarotene, bexarotene, and adapalene. These retinoids are commonly known as third-generation retinoids.

In yet another aspect, a retinoid belonging to the group known as second-generation retinoids (such as etretinate and acitretin) may be used in a composition of the invention for patients who suffer from the psoriatic condition.

In one aspect, the retinoid is present at a concentration below that which is typically utilized in topical formulations. For example, tazarotene or tazarotenic acid salt is included in a composition of the invention at a positive concentration less than 0.05 wt %.

In certain embodiments of the invention, a retinoid or a pharmaceutically acceptable salt or ester thereof is present in the composition at a positive concentration of less than 0.09% based on the weight of the composition. For example, the retinoid is present in the composition at a concentration in the range from about 0.001 to about 0.09 wt %, or from about 0.001 to about 0.08 wt %, or from about 0.001 to about 0.07 wt %, or from about 0.001 to about 0.06 wt %, or from about 0.01 to about 0.09 wt %, or from about 0.01 to about 0.08 wt %, or from about 0.01 to about 0.07 wt %, or from about 0.01 to about 0.06 wt %, or from about 0.001 to about 0.049 wt %, or from about 0.005 to about 0.045 wt %, or from about 0.005 to about 0.04 wt %, or from about 0.005 to about 0.03 wt %, or from about 0.005 to about 0.02 wt %, or from about 0.005 to about 0.01 wt %, or from about 0.01 to about 0.049 wt %, or from about 0.01 to about 0.045 wt %, or from about 0.02 to about 0.045 wt %, or from about 0.03 to about 0.045 wt %.

In another aspect, the composition described herein comprises a combination of: (a) halobetasol or a pharmaceutically acceptable salt or ester thereof; and (b) a tazarotene or a pharmaceutically acceptable tazarotenic acid salt or a pharmaceutically acceptable non-ethyl ester of tazarotenic acid, for treating a skin disease or condition associated with elevated levels of IL-17, or elevated levels of an IL-17 induced chemokine or effector (such as IL-6, IL-8, G-CSF, TNF-α, PGE2, and INF-γ); wherein (a) halobetasol or a pharmaceutically acceptable salt or ester thereof is present in the composition at a concentration in the range from about 0.005 to about 0.035 wt %, or from about 0.005 to about 0.025 wt %, or from about 0.01 to about 0.025 wt %; and (b) tazarotene or a pharmaceutically acceptable tazarotenic acid salt or a pharmaceutically acceptable non-ethyl ester of tazarotenic acid is present in the composition at a concentration in the range from about 0.001 to about 0.049 wt %, or from about 0.01 to about 0.049 wt %, or from about 0.01 to about 0.045 wt %, or from about 0.02 to about 0.04 wt %, or from about 0.02 to about 0.045 wt %, or from about 0.03 to about 0.045 wt %.

In still another aspect, the composition described herein comprises a combination of: (a) halobetasol or a pharmaceutically acceptable salt or ester thereof at a concentration of 0.01 wt %; and (b) tazarotene at a concentration of 0.045 wt %, or a pharmaceutically acceptable tazarotenic acid salt at a concentration that provides 0.045 wt % as tazarotene, for treating a skin disease or condition associated with elevated levels of IL-17 or elevated levels of an IL-17 induced chemokine or effector (such as IL-6, IL-8, G-CSF, TNF-α, PGE2, and INF-γ).

In still another aspect, the composition described herein comprises a combination of: (a) halobetasol or a pharmaceutically acceptable salt or ester thereof at a concentration of 0.01 wt %; and (b) tazarotene at a concentration of 0.045 wt %, or a pharmaceutically acceptable ester, other than ethyl ester, of tazarotenic acid at a concentration that provides 0.045 wt % as tazarotene, for treating a skin disease or condition associated with elevated levels of IL-17 or elevated levels of an IL-17 induced chemokine or effector (such as IL-6, IL-8, G-CSF, TNF-α, PGE2, and INF-γ). Non-limiting examples of non-ethyl esters of tazarotenic acid are disclosed herein above.

In yet another aspect, the composition described herein comprises a combination of: (a) halobetasol propionate at a concentration of 0.01 wt %; and (b) tazarotene at a concentration of 0.045 wt %, for treating a skin disease or condition associated with elevated levels of IL-17, or elevated levels of an IL-17 induced chemokine or effector (such as IL-6, IL-8, G-CSF, TNF-α, PGE2, and INF-γ).

In one aspect, a topical composition described herein useful in the methods of the invention is in the dosage form including, but not limited to, a gel, an emulsion (including lotion, cream, and milk), a shampoo, a foam, a suspension, a liquid, a spray, a paste or an ointment. In certain preferred embodiments, a topical composition described herein of the invention is an oil-in-water emulsion, in which an internal oil phase is dispersed in a continuous aqueous phase. Alternatively, water-in-oil-in-water, water-in-oil and oil-in-water-in-oil emulsions are also contemplated. The emulsion may be a macroemulsion, a microemulsion, or a nanoemulsion. Also included are other formulations in which an oil phase and a water phase coexist within the formulation, such as a multivesicular emulsion, which is not a true emulsion, disclosed in Espinoza, U.S. Pat. No. 6,709,663. Also included is a liposomal dispersion in a gel, cream or other dosage form. Also included are other formulations in which non-polar and polar liquid ingredients, with or without semipolar ingredients, coexist with the formulation.

In addition to the active ingredients as disclosed above, a composition of the invention comprises one or more dermatologically acceptable excipients, such as liquid oils, waxes viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents or cosolvents.

In one embodiment, the composition used in the methods of the invention is an oil-in-water emulsion. In one embodiment, the composition comprises in addition to the active agents: (i) a liquid oil component, and (ii) an aqueous component. In addition to the active agents, the liquid oil component, and the aqueous component, the composition, as noted above, can also include one or more dermatologically acceptable excipients, such as liquid oils, waxes viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents or cosolvents.

The liquid oil component of the composition includes one or more materials that are practically insoluble or insoluble in water and which are liquid at room temperature. For example, in one embodiment, the liquid oil component of the composition includes one or more materials that are practically insoluble or insoluble in water and which are liquid at room temperature of 22° C. The liquid oil component may be selected from one or more ingredients including, but not limited to, dicarboxylic acid esters (“DCAE”), monocarboxylic acid esters (“MCAE”), fish-liver oil, long-chain triglycerides (wherein each side chain has 14-18 carbons, such as peanut oil, sesame oil, coconut oil, sunflower oil, corn oil, olive oil, cotton seed oil, or derivatives thereof), propylene glycol diesters, medium-chain triglycerides (such as those wherein each side chains has 8-10 carbons; e.g., capric/caprylic acid triglycerides), hydrocarbons such as mineral oil, light mineral oil, squalene, and squalane, fatty alcohols (such as octyldodecanol and isostearyl alcohol), and fatty acids (such as isostearic acid and oleic acid).

The formulation may include other lipophilic liquids in an amount that is sufficient to be miscible with the dicarboxylic acid ester and/or monocarboxylic acid ester. The lipophilic liquid may be an emollient such as lanolin oil, mineral oil, light mineral oil, isostearic acid, squalene, octyldodecanol, fractionated coconut oil, cyclomethicone, or dimethicone.

In some embodiments, the liquid oil component comprises a dicarboxylic acid ester and light mineral oil. In some other embodiments, the liquid oil component comprises one or more long-chain triglycerides.

In addition to the liquid oil component, the formulation may contain water insoluble or practically insoluble ingredients that are not liquid at room temperature, but are soluble in the liquid oil component.

A DCAE that is suitable for use in the compositions of the invention has the formula R₁OOC—(CH₂)ₙ—COOR₂, wherein R₁ and R₂ are alkyl groups containing between 1 and 4 carbons or aryl groups and may be the same or may be different and wherein (CH₂)ₙ is a straight or branched chain and n is between 1 and 12. Examples of DCAEs containing one or more aryl groups are dibenzyl esters of dicarboxylic acids. A preferred dicarboxylic acid ester is diethyl sebacate, which has the formula CH₃CH₂OOC—(CH₂)₈—COOCH₂CH₃. Examples of other suitable dicarboxylic acid esters (where R₁ and R₂ are the same) are dimethyl, diethyl, dipropyl, diisopropyl, dibutyl and diisobutyl esters such as oxalate, malate, succinate, glutarate, adipate, pimelate, suberate, and azalate. Examples of suitable dicarboxylic acid esters (where R₁ is different from R₂) are methyl ethyl, methyl propyl, methyl butyl, methyl isopropyl, ethyl propyl, ethyl butyl, ethyl isopropyl, and propyl butyl esters such as oxalate, malate, succinate, glutarate, adipate, pimelate, suberate, azalate, and sebacate.

Alternatively, or in combination with the DCAE, the compositions of the invention can also contain a MCAE. The MCAE that is suitable for the invention has the formula CH₃—(CH₂)ₙ—COOR₁, wherein R₁ is an alkyl group containing between 1 and 4 carbons or an aryl group, and wherein (CH₂)ₙ is straight or branched chain and n is between 1 and 12. Examples of such monocarboxylic acid esters include methyl, ethyl, propyl, isopropyl, butyl, or an aryl such as benzyl formate, acetate, propionate, butyrate, valerate, laurate, myristate, palmitate, and stearate. Examples of preferred monocarboxylic acid esters are isopropyl palmitate and isopropyl myristate.

The liquid oil phase can beneficially be used to dissolve one or more of the active ingredients within the emulsion or other type of composition of the invention. In one embodiment, the corticosteroid, such as halobetasol propionate, and the retinoid, such as tazarotene, are both dissolved in the liquid oil phase within the composition at room temperature.

In another embodiment, the corticosteroid is dissolved in the liquid oil phase, and the retinoid is suspended within the composition at room temperature. In yet another embodiment, the retinoid is dissolved in the liquid oil phase, and the corticosteroid is suspended within the composition at room temperature. In the case wherein the retinoid or the corticosteroid is suspended in the composition, it is preferred that the suspended active ingredient be micronized, namely that the mean particle size is preferably about 25 microns in diameter or less.

In addition to the active ingredients, the liquid oil component, and the aqueous component, which have been described above, a composition of the invention comprises one or more dermatologically acceptable excipients, such as liquid oils, waxes viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents or cosolvents.

The composition of the invention may desirably contain a thickening agent to provide viscosity so that the formulation may be provided in the form of a lotion, gel, cream, or ointment. Preferably, but not necessarily, the thickening agent is miscible or soluble in an aqueous fluid. Non-limiting examples of suitable thickening agents include, but are not limited to, acacia, alginic acid and its salts, hyaluronic acid and its salts, carbomers (also known as carboxy vinyl polymers, which are cross-linked polyacrylic acid), carboxymethylcellulose, ethylcellulose, gelatin, hydroxyethyl cellulose, hydroxypropyl cellulose, hydroxypropylmethyl cellulose, methylcellulose, poloxamers, polyvinylpyrrolidone, polyvinyl alcohol, tragacanth, xanthan gum, magnesium aluminum silicate, and bentonite. The thickening agent may also reside in the liquid oil component or lipophilic portion of the formulation. Examples of suitable lipophilic thickening agents include, but are not limited to, cetyl alcohol, stearyl alcohol, glyceryl stearate, white beeswax, microcrystalline wax, hydrogenated polyisobutane polymers, and emulsifying wax.

Thickening agents suitable for use in the compositions of the invention include carbomers, such as Carbopol® and polycarbophil (The Lubrizol Corporation, Wickliffe, Ohio). Carbopol® homopolymers are polymers of acrylic acid crosslinked with allyl sucrose or allylpentaerythritol. Carbopol® copolymers are polymers of acrylic acid and C₁₀-C₃₀ alkyl acrylate crosslinked with allylpentaerythritol. Carbopol® interpolymers are carbomer homopolymers or copolymers that contain a block copolymer of polyethylene glycol and a long chain alkyl acid ester. Noveon® polycarbophil is a polymer of acrylic acid crosslinked with divinyl glycol.

A surfactant or emulsifier can also be included in the compositions of the invention, if desired or required. Pharmaceutically acceptable anionic, cationic, or non-ionic surfactants may be included in a composition of the invention. Non-ionic surfactants are preferred. Non-limiting examples of non-ionic surfactants include, but are not limited to, Octoxynol (also known as Macrogol tetramethylbutylphenyl ether, octylphenoxy polyethoxyethanol, or polyoxyethylene octylphenyl ether), such as Octoxynol 1, 3, 5, 8, 9, 10, 12, 13, 16, 30, 40, 70 (wherein the number indicates the number of repeating oxyethylene units), or other Octoxynols that comprise different numbers of repeating units of oxyethylene in the side chain, sorbitan esters (such as sorbitan monooleate and sorbitan monostearate, commonly known by their trade names Span 80 and Span 60), polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108), or poloxamines (synthetic block polymers of ethylene oxide and propylene oxide attached to ethylene diamine, such as those commonly known by their trade names of Tetronic®; e.g., Tetronic® 1508 or Tetronic® 908, etc.), other nonionic surfactants such as Brij® (polyoxyethylene alkyl ether having a formula of CH₃—(CH₂)₁₀₋₁₆—(O—C₂H₄)₁₋₂₅—OH), Myrj® (stearic acid esterified with polyoxyethylene having 40-100 repeating oxyethylene units), and long chain fatty alcohols (e.g., oleyl alcohol, stearyl alcohol, myristyl alcohol, docosahexaenoyl alcohol, etc.) with carbon chains having about 12 or more carbon atoms (e.g., such as from about 12 to about 24 carbon atoms).

In addition, polymeric emulsifiers such as those known under the trade name Pemulen™ (The Lubrizol Corporation, Wickliffe, Ohio) can also be included in the compositions of the invention. These are polymers of acrylic acid, modified by long chain (C₁₀-C₃₀)alkyl acrylates, and crosslinked with allylpentaerythritol.

An anionic emulsifier can also be included in the compositions of the invention. Suitable anionic emulsifiers include, but are not limited to, sodium or potassium oleate, triethanolamine stearate, sodium lauryl sulfate, sodium dioctyl sulfosuccinate, and sodium docusate. Less preferred are cationic emulsifiers such as quaternary ammonium salts. Still other emulsifiers include, but are not limited to, glyceryl monostearate, polyoxyethylene monooleate, polyoxyethylene monostearate, polyoxyethylene monolaurate, potassium oleate, sodium lauryl sulfate, sodium oleate, sorbitan monolaurate, sorbitan monopalmitate, sorbitan monostearate, sorbitan tristearate, sorbitan monooleate, sorbitan trioleate, triethanolamine oleate, polyoxyethylene sorbitan monolaurate, polyoxyethylene sorbitan monopalmitate, polyoxyethylene sorbitan monostearate, polyoxyethylene sorbitan tristearate, polyoxyethylene sorbitan monooleate, and polyoxyethylene sorbitan trioleate.

The compositions of the invention desirably include a dermatologically acceptable humectant. Suitable humectants include, but are not limited to, glycerin, sorbitol, hexylene glycol, propylene glycol, or urea. In addition, the formulation may contain an emollient such as petrolatum, lanolin, mineral oil, light mineral oil, stearic acid, cyclomethicone, or dimethicone. Chelating agents such as EDTA and its salts may also be included in a formulation of the invention.

In one aspect, a composition of the invention comprises the ingredients at the concentrations shown in Table 2.

TABLE 2
Compositions of the Invention for Treating Diseases and
Conditions Associated with Elevated Levels of IL-17

| | Concentration (wt %) | | |
|---|---|---|---|
| Ingredient | Range 1 | Range 2 | Range 3 |
| Class-1 Corticosteroid | 0.001-0.049 | 0.005-0.035 | 0.01-0.025 |
| Tazarotene or | 0.01-0.049 | 0.02-0.045 | 0.03-0.045 |
| Tazarotenic Acid | | | |
| Compound | | | |
| Emollient, Solvent, | 0.5-40 | 1-25 | 2-20 |
| and/or Thickener | | | |
| Emulsifier | 0.25-10 | 0.5-7 | 1-5 |
| Humectant | 0-15 | 2-12 | 10 |
| Polymeric Thickener | 0.05-2 | 0.1-1.5 | 0.3-1 |
| Pharmaceutical Aids | q.s. | q.s. | q.s. |
| Purified water | q.s. to 100 | q.s. to 100 | q.s. to 100 |

Non-limiting examples of compositions that are useful in the methods of the invention are shown in Table 3.

TABLE 3
Some Emulsion Compositions of the Invention for Treating Diseases
and Conditions Associated with Elevated Levels of IL-17

| | | Concentration (wt %) | | | |
|---|---|---|---|---|---|
| | | | | | Preferred |
| Ingredient | Function | Range 1 | Range 2 | Range 3 | embodiment |
| Halobetasol | Class 1 | 0.001-0.04 | 0.005-0.03 | 0.005-0.02 | 0.01 |
| propionate | Corticosteroid | | | | |
| Tazarotene | Retinoid | 0.02-0.049 | 0.03-0.049 | 0.04-0.049 | 0.045 |
| Diethyl sebacate | Liquid Oil & | 1-5 | 2-4 | 2.5-3.5 | 2.97 |
| | Solvent | | | | |
| Light mineral oil | Liquid Oil & Co- | 5-15 | 5-10 | 7.5-8.5 | 8.03 |
| | Solvent | | | | |
| Sorbitan | Surfactant/ | 0.01-1 | 0.02-0.5 | 0.05-0.2 | 0.1 |
| monooleate | Emulsifying | | | | |
| | Agent | | | | |
| Sorbitol | Humectant | 5-15 | 7-12 | 10-11 | 10.7 |
| solution, 70% | | | | | |
| Methyl paraben | Antimicrobial | 0.05-0.3 | 0.1-0.3 | 0.1-0.2 | 0.17 |
| | Preservative⁽¹⁾ | | | | |
| Propyl paraben | Antimicrobial | 0.01-0.1 | 0.01-0.05 | 0.02-0.04 | 0.03 |
| | Preservative⁽¹⁾ | | | | |
| Edetate | Chelating | 0.02-0.1 | 0.02-0.7 | 0.03-0.06 | 0.05 |
| disodium | Agent⁽¹⁾ | | | | |
| dihydrate | | | | | |
| Carbomer | Emulsifying | 0.1-1 | 0.2-0.7 | 0.3-0.5 | 0.4 |
| copolymer type | Agent | | | | |
| B (e.g., Pemulen ™ | | | | | |
| TR-1) | | | | | |
| Carbomer | Thickener | 0.2-1.5 | 0.3-1 | 0.5-0.7 | 0.6 |
| homopolymer | | | | | |
| type A (e.g., | | | | | |
| Carbomer 981) | | | | | |
| Sodium | pH-adjusting | q.s. to pH | q.s. to pH | q.s. to pH | q.s. to pH |
| hydroxide, 10% | Agent⁽¹⁾ | of 5.5 ± 0.5 | of 5.5 ± 0.5 | of 5.5 ± 0.5 | of 5.5 ± 0.5 |
| Solution | | | | | |
| Purified water | Carrier | q.s. to 100 | q.s. to 100 | q.s. to 100 | q.s. to 100 |

Note:
⁽¹⁾These ingredients are broadly classified as Pharmaceutical Aids.

In one embodiment, the composition used in the methods of the invention is preferably the composition set forth in Table 4:

TABLE 4
Preferred Composition - IDP-118

| | | IDP-118 (Invention |
|---|---|---|
| | | Embodiment) |
| Ingredient | Function | Concentration (wt %) |
| Halobetasol propionate, | Active | 0.01 |
| micronized | | |
| | Ingredient | |
| Tazarotene | Active | 0.045 |
| | Ingredient | |
| Diethyl sebacate | Emollient & Solvent | 2.97 |
| Light mineral oil | Emollient & Solvent | 8.03 |
| Sorbitan monooleate | Surfactant/ | 0.1 |
| | Emulsifying Agent | |
| Sorbitol solution, 70% | Humectant | 10.7 |
| Methyl paraben | Antimicrobial | 0.17 |
| | Preservative | |
| Propyl paraben | Antimicrobial | 0.03 |
| | Preservative | |
| Edetate disodium | Chelating Agent | 0.05 |
| dihydrate | | |
| Carbomer copolymer | Emulsifying Agent | 0.4 |
| type B (Pemulen ™ TR-1) | | |
| Carbomer homopolymer | Thickener | 0.6 |
| type A (Carbomer 981) | | |
| Sodium hydroxide | pH-adjusting agent | q.s. to pH of 5.5 ± 0.5 |
| Purified water | Solvent | q.s. to 100 |

A lotion having a composition as shown in the last column of Table 2 was prepared as follows.

A separate aqueous phase was made. In a manufacturing vessel equipped with a mixing implement (such as a propeller) and temperature control, purified water and disodium edetate dihydrate were combined and the mixture was agitated until a clear solution is achieved. Sorbitol, methylparaben, and propylparaben were then added to the mixture. The mixture was continuously mixed and was heated to approximately 75° C. The mixture was agitated until a solution was obtained. The mixture was then removed from the heat source and allowed to cool to below 40° C. with continued mixing. With continuous mixing, Carbopol® 981 was added to the mixture and dispersed. Mixing continued until Carbopol® 981 was fully dispersed and hydrated.

A separate oil phase was made. In a vessel equipped with a mixing implement such as a propeller, diethyl sebacate, halobetasol propionate, and tazarotene were combined. The mixture was agitated until a solution was achieved. With continuous mixing, light mineral oil and sorbitan monooleate were added. Mixing is continued until a solution is obtained.

In a separate vessel, a 1N solution of sodium hydroxide is prepared.

With high speed mixing, the oil phase containing the active ingredients (halobetasol propionate and tazarotene) was added to the aqueous phase. Pemulen™ TR-1 was slowly added into the mixture. Mixing was continued until a homogeneous emulsion was obtained. Mixing speed was decreased and mixing continued for an additional time of 10 minutes to 1 hour. With continuous mixing, an appropriate amount of the sodium hydroxide solution was added incrementally to obtain a pH of 5.5±0.5. Mixing continued further until a homogeneous lotion was obtained, such as for 30 minutes to 3 hours.

Alternatively, Pemulen™ TR-1 may be added into the aqueous phase when it was made.

B. Methods for Treating Diseases and Conditions Associated with Elevated Levels of IL-17

In another aspect, the invention provides a method for treating a skin disease or condition associate with IL-17. In one embodiment, the skin disease or condition to be treated is associated with an elevated level of IL-17 or an elevated level of an IL-17 induced chemokine or effector (such as IL-6, IL-8, G-CSF, TNF-α, PGE2, and INF-γ). Methods for determining the levels of IL-17 or any of its chemokines or effectors in a subject are well-known to and used by those of ordinary skill in the art. Typically, a tissue or biological fluid sample is obtained from the patient and is subject to ELISA with commercially available antibodies or kits (e.g., Quantikine IL-17 ELISA; R&D Systems, Abington, UK). Levels of IL-17 or any of its chemokines or effectors are considered elevated if the level is above that which is considered normal for subjects not having or suffering from the disease or condition. Those of skill in the art will readily be able to determine if the level of IL-17 or any of its chemokines or effectors is elevated. For instance, commercially available assays or assays similar to those used in the Examples, below, can be used to determine IL-17 levels, to determine if IL-17 levels are elevated, and to determine if the compositions of the invention can be used to reduce or inhibit IL-17 levels. Similarly, commercially available assays can be used to determine levels of an IL-17 induced chemokines or effectors, to determine if levels of an IL-17 induced chemokines or effectors are elevated, and to determine if the compositions of the invention can be used to reduce or inhibit of levels of an IL-17 induced chemokines or effectors. In addition to the assays disclosed in the Examples, commercially available antibodies and kits are available for IL-6, IL-8, G-CSF, TNF-α, IL-1β, PGE2, and IFNγ. Other suitable assays for measuring levels of IL-17 or any of its chemokines or effectors are disclosed in U.S. Patent Application Publication No. 2015/0005319, the teachings of which are incorporated herein by reference.

In one embodiment, the skin disease or condition associate with IL-17 is an inflammatory, autoimmune, or oncological (proliferative) skin disease or condition. Exemplary inflammatory skin diseases or conditions that can be treated using the compositions of the invention include, but are not limited to, atopic dermatitis, pustular psoriasis, acne keloidalis nuchae (ACN), acne vulgaris, psoriatic arthritis, dermatitis herpetiformis (DH), bullous pemphigoid (BP), pemphigus vulgaris (PV), ankylosing spondylitis, pityriasis rubra pilaris (PRP), and hidradenitis suppurative. Exemplary autoimmune skin diseases or conditions that can be treated using the compositions of the invention include, but are not limited to, vitiligo and alopecia areata. Exemplary oncological skin diseases or conditions that can be treated using the compositions of the invention include, but are not limited to, non-melanoma skin cancer (NMSC), melanoma, cutaneous T-cell lymphoma (CTCL). In one embodiment, the skin disease or condition to be treated is cutaneous T-cell lymphoma (CTCL). In another embodiment, the skin disease or condition to be treated is acne keloidalis nuchae (ACN). In another embodiment, the skin disease or condition to be treaed is pustular psoriasis.

The method comprises topically applying to an affected area of the body of a subject suffering from the disease or condition associated with elevated IL-17 any one of the compositions of the invention, as disclosed herein, one or more times per day for a period of time sufficient to treat such skin disease or condition. For example, such a period of time may be 1 to 30 days or longer as needed. For example, such a period of time may be one week, two weeks, four weeks, eight weeks, twelve weeks, or longer as needed. For example, a composition of the invention is applied topically to affected areas of the body once per day for 7-14 days. Alternatively, it may be applied two or three times per day for 7-14 days. Alternatively, it may be applied once per day for one week to six months or once a day for one week to 12 months or longer. For example, it may be applied once per day for two weeks, four weeks, eight weeks, or twelve weeks. In one embodiment, the treatment may be stopped for 1-7 days (e.g., 2, 3, 4, 5, 6, or 7 days) after an extended treatment period before it is resumed for another extended treatment period. Such an extended period may be 7 days, 7-14 days, 7-21 days, 7-30 days, or longer before more treatment is needed or desired.

In yet another aspect, the invention provides a method of treating a skin disease or condition associate with elevated levels of IL-17 or elevated levels of an IL-17 induced chemokine or effector (such as IL-6, IL-8, G-CSF, TNF-α, PGE2, and INF-γ) topically with a pharmaceutical composition comprising a combination of: (a) halobetasol or a pharmaceutically acceptable salt or ester thereof; and (b) a tazarotene or a pharmaceutically acceptable tazarotenic acid salt, or ester; wherein each of: (a) halobetasol or a pharmaceutically acceptable salt, or ester thereof; and (b) tazarotene or a pharmaceutically acceptable tazarotenic acid salt, or ester is present in the composition at a positive concentration of less than 0.05% based on the weight of the composition. In some embodiments, the clinical efficacy of the combination is greater than that of either the halobetasol component or the tazarotene component at the same concentration when used alone.

In yet another aspect, the invention provides a method of treating a skin disease or condition associate with elevated levels of IL-17 or elevated levels of an IL-17 induced chemokine or effector (such as IL-6, IL-8, G-CSF, TNF-α, PGE2, and INF-γ) topically with pharmaceutical composition comprising a combination of: (a) halobetasol or a pharmaceutically acceptable salt, or ester thereof; and (b) a tazarotene or a pharmaceutically acceptable tazarotenic acid salt, or ester; wherein each of: (a) halobetasol or a pharmaceutically acceptable salt, or ester thereof; and (b) tazarotene or a pharmaceutically acceptable tazarotenic acid salt, or ester is present in the composition at a positive concentration of less than 0.05% based on the weight of the composition. In some embodiments the clinical success rate of the combination is synergistic compared to the clinical success rate of the halobetasol component at the same concentration used alone plus the clinical success rate of the tazarotene component used alone at the same concentration.

In yet another aspect, the invention provides topical pharmaceutical compositions comprising a combination of: (a) halobetasol or a pharmaceutically acceptable salt, or ester thereof; and (b) a tazarotene or a pharmaceutically acceptable tazarotenic acid salt, or ester thereof, and methods using the same, for treating a skin disease or condition associate with elevated levels of IL-17 or elevated levels of an IL-17 induced chemokine or effector (such as IL-6, IL-8, G-CSF, TNF-α, PGE2, and INF-γ); wherein the pharmaceutical composition is a cream, lotion, ointment, gel, shampoo, suspension, paste, plaster, foam, spray or solution. Exemplary compositions are illustrated in Table 5.

In yet another aspect, the invention provides a method of treating a skin disease or condition associate with elevated levels of IL-17 or elevated levels of an IL-17 induced chemokine or effector (such as IL-6, IL-8, G-CSF, TNF-α, PGE2, and INF-γ) topically with pharmaceutical composition comprising a combination of: (a) halobetasol or a pharmaceutically acceptable salt, or ester thereof; and (b) a tazarotene or a pharmaceutically acceptable tazarotenic acid salt, or ester; wherein the composition is applied at least once daily for 4 weeks, such as for 6 weeks or 8 weeks.

In yet another aspect, the invention provides a method of treating a skin disease or condition associate with elevated levels of IL-17 or elevated levels of an IL-17 induced chemokine or effector (such as IL-6, IL-8, G-CSF, TNF-α, PGE2, and INF-γ) topically with pharmaceutical composition comprising a combination of: (a) halobetasol or a pharmaceutically acceptable salt, or ester thereof; and (b) a tazarotene or a pharmaceutically acceptable tazarotenic acid salt, or ester; wherein the composition is applied once daily for more than 2 weeks, such as 4 weeks, for 6 weeks or for 8 weeks without any serious adverse events (side effects).

In yet another aspect, a composition of the invention that can be used in any of the methods disclosed herein can be in the form of a solution, a spray, a milk, a foam, or an ointment. Non-limiting examples of these compositions are shown in Table 5.

TABLE 5
Examples of Various Dosage Forms of Compositions of the Invention

| | Solution | Spray | Milk | Foam | Ointment |
|---|---|---|---|---|---|
| Ingredient | Concentration (% wt) | | | | |
| Corticosteroid | 0.001-0.049 | 0.001-0.049 | 0.001-0.049 | 0.001-0.049 | 0.001-0.049 |
| Tazarotene or tazarotenic acid | 0.01-0.049 | 0.01-0.049 | 0.01-0.049 | 0.01-0.049 | 0.01-0.049 |
| Humectant | 0-15.0 | 0-15.0 | 0-15.0 | 0-15.0 | — |
| Antimicrobial preservative | 0.01-0.4 | 0.01-0.4 | 0.01-0.4 | 0.01-0.4 | — |
| Chelating agent | 0.02-0.1 | 0.02-0.1 | 0.02-0.1 | 0.02-0.1 | — |
| Emollients/Lipophilic Solvents | qsad 100 | 0.5-50.0 | 0.5-50.0 | 0.5-50.0 | 0.5-50.0 |
| Volatile solvent | — | qsad 100 | — | — | — |
| Non-ionic polyethoxylated compounds | — | — | — | — | — |
| Non-ionic sorbitan compounds | — | — | 0.5-5.0 | — | 0.5-5.0 |
| Polyoxyethylene fatty ethers | — | — | — | 0.5-10.0 | — |
| Fatty alcohols | — | — | — | 1-8.0 | 1.0-10.0 |
| Petrolatum | — | — | — | — | qsad 100 |
| Pharmaceutical aids (pH adjustment) | — | — | 0.1-5.0 | 0.1-5.0 | — |
| Purified water | — | — | qsad 100 | qsad 100 | — |

In still another aspect, a composition of the invention may be used in conjunction with another method of treating a skin disease or condition associate with elevated levels of IL-17 or elevated levels of an IL-17 induced chemokine or effector (such as IL-6, IL-8, G-CSF, TNF-α, PGE2, and INF-γ), such as phototherapy (e.g., with ultraviolet light).

In yet another aspect, a composition of the invention may be used in conjunction with another medicament for treating a skin disease or condition associate with elevated levels of IL-17 or elevated levels of an IL-17 induced chemokine or effector (such as IL-6, IL-8, G-CSF, TNF-α, PGE2, and INF-γ). Such other medicament may be an anti-TNF-α agent (e.g., infliximab, etanercept, adalimumab, or golimumab), an agent targeting the Th17/IL-23 axis (e.g., ustekinumab or briakinumab), a protein kinase C inhibitor (e.g., AEB071), a mitogen-activated protein kinase inhibitor (e.g., BMS-582949), a FMS-like tyrosine kinase inhibitor (e.g., lestaurtinib), a Janus kinase inhibitor tofacitinib, ASP-015K, or INCB018424), a phosphodiesterase 4 inhibitor (e.g., apremilast, AN2728, or MK0873), a nerve growth factor inhibitor (e.g., CF101), an anti-folate agent (e.g., methotrexate, aminopterin, or BCX-4208), a calcineurin inhibitor (e.g., cyclosporine), an anti-angiogenic agent (e.g., anti-VEGF antibody or soluble VEGFR), or a vitamin D analog or derivative. Such other medicament may be administered to a patient at substantially the same time or at a different time. Such other medication may be administered topically, orally, or by injection or infusion.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker. As used herein, the phrase “a subject in need thereof” refers to a subject, as described infra, that suffers from, or is at risk for, a pathology to be prophylactically or therapeutically treated with a compound or salt described herein.

The term “effective amount” or “therapeutically effective amount” refers to that amount of a compound or salt or composition as described herein that is sufficient to effect the intended application including but not limited to treatment of the disease or condition associated with IL-17 treatment. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein or inhibition of IL-17. The specific dose can vary depending on the particular compounds chosen, the composition selected, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample. It will be appreciated by those of skill in the art that in addition to treating a skin disease or condition associated with IL-17, the compositions and methods of the invention can be used to prevent or delay the onset of the skin disease or condition in a subject believed to be susceptible to such skin disease or condition.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

While the present disclosure shows and describes a number of exemplary embodiments, it will be manifest to those skilled in the art that various further modifications may be made without departing from the spirit and scope of the underlying inventive concept and that the same is not limited to particular compositions, processes, methods, or structures herein shown and described. All patents and publications referred to herein are incorporated by reference in their entireties.

### EXAMPLES

### Example 1: Disease Modification of Tazarotene/Halobetasol Lotion

Objective: The objective of this study was to demonstrate disease modification through anti-inflammatory effects (reduction in TNF-«, IL-17A) of tazarotene/halobetasol lotion in patients with mild to moderate plaque type psoriasis as compared to control.

### 1. PROTOCOL SYNOPSIS

| Title of Study: | A Demonstration of the Anti-Inflammatory Effects of 0.045% | |
|---|---|---|
| | Tazarotene/0.01% Halobetasol Lotion in the Treatment of Psoriasis | |
| Study Period: | 12 weeks or 4 weeks after psoriasis treatment plaque clearing | |
| Test Product, Dose | 0.01% Halobetasol and 0.045% Tazarotene Lotion (Duobrii product) | |
| and Mode of | Applied once daily to designated target plaques at bedtime. | |
| Administration: | | |
| Objective: | The objective was to demonstrate disease modification through anti- | |
| | inflammatory effects (reduction in TNF-α, IL-17A) of | |
| | tazarotene/halobetasol lotion in patients with mild to moderate plaque | |
| | type psoriasis as compared to control. | |
| Design: | Male or female subjects with mild to moderate plaque type psoriasis | |
| | presented to the research center for evaluation for study entry. They | |
| | were advised to present to the research center with clean skin and no | |
| | topical products applied to the body, including medications, | |
| | moisturizers, sunscreens, etc. If found to be suitable by meeting all | |
| | inclusion criteria and none of the exclusion criteria, subjects underwent | |
| | selection of 2 target plaques by the dermatologist investigator. Each | |
| | plaque was graded for erythema, scaling, and induration. An | |
| | Investigational Global Assessment (IGA) score was assigned for each | |
| | plaque. Plaques were an IGA of mild (2) or moderate (3) to qualify for | |
| | inclusion. The subjects also assessed each plaque for redness, | |
| | thickness, and scaling. Each of the plaques was photographed. | |
| | The subjects underwent D-squame tape strip removal of each of the 2 | |
| | target psoriasis plaques. One plaque served as the | |
| | tazarotene/halobetasol treatment plaque and the other served as the | |
| | untreated control. 10 D-squames were obtained with from each plaque. | |
| | 5 tape strips were placed in each glass jar. One jar contained the even | |
| | tape strips and the other jar contained the odd tape strips. 10 D- | |
| | squames were also taken from normal skin to serve as a negative | |
| | control in an individual without psoriasis. 5 tape strips were placed in | |
| | each glass jar. One jar contained the even tape strips and the other jar | |
| | contained the odd tape strips. The D-squames were frozen in a −80 C. | |
| | freezer for later assessment. Five tape strips were analyzed together to | |
| | obtain adequate material for LCMS analysis. Two specimens jars were | |
| | prepared for from each of the 2 target plaques. One jar was analyzed | |
| | with the second jar serving as a back up. It has been determined from | |
| | the pilot study that the inflammatory mediator amount is fairly | |
| | consistent at skin depths reached by 10 tape strips. | |
| | The subjects had a number assigned to each of the treatment plaques. | |
| | The numbers were recorded on a body map. Two copies were | |
| | prepared. One copy remained at the research center and the second | |
| | copy was provided to the subject. Each subject received one tube of | |
| | tazarotene/halobetasol lotion for application to the treated plaque. The | |
| | second plaque functioned as an untreated control site. | |
| | Subjects were provided with a diary. They were instructed to apply the | |
| | study product once daily at bedtime to the treated plaque only. | |
| | Following application of the tazarotene/halobetasol lotion to the treated | |
| | plaque once daily at bedtime, both the treated and untreated plaques | |
| | received moisturizer application once daily. Care was taken to apply | |
| | the proper product to the proper plaque. Subjects were instructed to | |
| | return to the research center in 2 weeks. A reminder text for | |
| | compliance was provided prior to the week 2 visit. | |
| | Subjects returned to the research center at week 2. The dermatologist | |
| | investigator evaluated each plaque for erythema, scaling, and | |
| | induration. An Investigational Global Assessment (IGA) score was | |
| | assigned for each plaque. The subjects also assessed each plaque for | |
| | redness, thickness, and scaling. Each of the plaques was photographed. | |
| | The subjects underwent D-squame tape strip removal of 10 tape strips | |
| | for each of the 2 target psoriasis plaques. Five tape strips were placed | |
| | in each jar with one jar receiving the even tape strips and the other jar | |
| | receiving the odd tape strips. The D-squames were frozen in a −80° C. | |
| | freezer for later assessment. Subjects were asked to returned to the | |
| | research center at week 4 or sooner if the treated psoriasis plaque has | |
| | cleared (IGA = 0). If the treated plaque has cleared, subjects contacted | |
| | the research center and 4 weeks after treatment plaque clearing, | |
| | underwent a final tape stripping procedure and completed their study | |
| | participation. A reminder text for compliance was sent prior to the | |
| | week 4 visit. | |
| | Subjects returned to the research center at week 4. The dermatologist | |
| | investigator evaluated each plaque for erythema, scaling, and | |
| | induration. An Investigational Global Assessment (IGA) score was | |
| | assigned for each plaque. The subjects also assessed each plaque for | |
| | redness, thickness, and scaling. Each of the plaques was photographed. | |
| | The subjects underwent D-squame tape strip removal 10 times for each | |
| | of the 2 target psoriasis plaques. Five tape strips were placed in each | |
| | jar with one jar containing the even tape strips and the other jar | |
| | containing the odd tape strips. The D-squames were frozen in a −80 C. | |
| | freezer for later assessment. Subjects who experienced clearing | |
| | (IGA = 0) of the treatment plaque discontinued treatment and returned to | |
| | the research at week 8. Subjects who had remaining psoriasis at the | |
| | site of the treatment plaque continued treatment as specified previously | |
| | and returned to the treatment center at week 8. A reminder text for | |
| | compliance was sent prior to the week 8 visit. | |
| | Subjects returned to the research center at week 8. The dermatologist | |
| | investigator evaluated each plaque for erythema, scaling, and | |
| | induration. An Investigational Global Assessment (IGA) score was | |
| | assigned for each plaque. The subjects also assessed each plaque for | |
| | redness, thickness, and scaling. Each of the plaques was photographed. | |
| | The subjects underwent D-squame tape strip removal of each of the 2 | |
| | target psoriasis plaques. 10 tape strips were removed from each | |
| | plaque. Five tape strips were placed in each jar with the even tapes | |
| | placed in one jar and the odd tapes placed in the second jar. The D- | |
| | squames were frozen in a −80 C. freezer for later assessment. Subjects | |
| | who experienced clearing (IGA = 0) at week 8 discontinued treatment | |
| | and returned to the research at week 12. Subjects who did not have an | |
| | IGA = 0 or clearing of the treatment plaque continued study drug | |
| | application and returned to the research center at week 12. | |
| | Subjects returned to the research center at week 12. The dermatologist | |
| | investigator evaluated each plaque for erythema, scaling, and | |
| | induration. An Investigational Global Assessment (IGA) score was | |
| | assigned for each plaque. The subjects also assessed each plaque for | |
| | redness, thickness, and scaling. Each of the plaques was photographed. | |
| | The subjects underwent D-squame tape strip removal of each of the 2 | |
| | target psoriasis plaques 10 times to procure 10 tape strips. Five tape | |
| | strips were placed in each jar with the even tape strips placed in one jar | |
| | and the odd tape strips placed in a second jar. The D-squames were | |
| | frozen in a −80 C. freezer for later assessment. All subjects completed | |
| | their study participation. | |
| | The length of participation for the subjects depended on the presence of | |
| | an IGA = 0 at the treated target plaque. Subjects who were clear at week | |
| | 4 discontinued treatment and returned for study completion at week 8. | |
| | Subjects who were clear at week 8 discontinued treatment and returned | |
| | for study completion at week 12. If clearing occurs in between visits, | |
| | the subjects contacted the research center and the date of clearing was | |
| | noted and treatment discontinued. Subjects returned for the final tape | |
| | stripping and study completion 4 weeks after clearing had occurred. | |
| Study Population: | Healthy male or non-pregnant female subjects 18+ years of age with | |
| | plaque type psoriasis | |
| Number of Subjects: | 10 subjects | |
| Inclusion Criteria: | The following represented the inclusion criteria: | |
| | 1. | Males or non-pregnant females 18+ years of age. |
| | 2. | Plaque type mild to moderate psoriasis suitable for topical |
| | | treatment. |
| | 3. | The presence of 2 plaques suitable for tape stripping |
| | 4. | Subjects must be willing to allow a series of tape pieces to be |
| | | pressed and removed from 2 target psoriasis plaques. |
| | 5. | Subjects must be in general good health as determined from a |
| | | medical history. |
| | 6. | Subjects must read and sign the informed consent form after the |
| | | nature of the study has been fully explained. |
| Exclusion Criteria: | The following represented the exclusion criteria: | |
| | 1. | Subjects with known allergies or sensitivities to ingredients |
| | | contained in the test products. |
| | 2. | Subjects with an allergy to latex or adhesives. |
| | 3. | Subjects with pustular or erythrodermic psoriasis. |
| | 4. | Subjects who are pregnant or nursing or planning to become |
| | | pregnant during the course of the study. |
| | 5. | Subjects who are currently participating in any other clinical |
| | | study (i.e., dermal patch, use tests, investigational drug or |
| | | devices, etc.). |
| | 6. | Subjects viewed by the investigator as not being able to |
| | | completed the study. |
| | 7. | Subjects using any type of lotion, medication, or other topical |
| | | product to the psoriasis plaques. |

| | |
|---|---|
| Endpoint: | Primary Efficacy Endpoint: |
| | The primary efficacy endpoint was the decrease in TNF-α and/or IL- |
| | 17A after achieving an IGA of 0 with tazarotene/halobetasol lotion as |
| | compared to control. |
| | Secondary Efficacy Endpoint: |
| | The secondary efficacy endpoint was the decrease or maintained |
| | decrease in TNF-α and/or IL-17A after treatment for 4 weeks with |
| | tazarotene/halobetasol lotion as compared to the no treatment control. |
| Measures: | Investigator Assessments: |
| | Plaque Assessments: The dermatologist investigator assessed |
| | erythema, induration, and scaling at baseline, week 2, week 4, week 8, |
| | and 12. Each of the 2 target plaques was assessed separately. |
| | Plaque IGA Assessments: The dermatologist investigator assessed |
| | plaque IGA at baseline, week 2, week 4, week 8, and week 12. Each of |
| | the 2 target plaques was assessed separately. |
| | Subject Assessments: |
| | The subjects assessed plaque redness, thickness, and scaling at |
| | baseline, week 2, week 4, week 8, and week 12. Each of the 2 target |
| | plaques was assessed separately. |
| | ELISA: |
| | 10 D-squames were removed from each of the 2 target plaques at |
| | baseline, week 2, week 4, week 8, and week 12. Five D-squames were |
| | placed in each jar with the even tape strips placed in one jar and the |
| | odd tape strips placed in a second jar. One jar was analyzed and the |
| | other jar served as a backup. Five tape strips provided enough material |
| | for analysis. The tape strips were solvent treated to remove the skin |
| | scale. The mixture was clarified with centrifugation and the |
| | supernatant were analyzed with ELISA to determine the amount of |
| | TNF-α and IL-17A. This was repeated for the specimens from each of |
| | the 2 target plaques (tazarotene/halobetasol lotion and untreated). The |
| | amount of TNF-α and IL-17A were compared for the 2 target plaques |
| | at each collection time point (tazarotene/halobetasol lotion and |
| | untreated). |
| | Photography: |
| | High quality jpeg images were taken of all target plaques at baseline, |
| | week 2, week 4, week 8, and week 12. |
| | Note: The length of participation for the subjects depended on the |
| | presence of an IGA = 0 at the treated target plaque. Subjects who were |
| | clear at week 4 discontinued treatment and returned for study |
| | completion at week 8. Subjects who were clear at week 8 discontinued |
| | treatment and returned for study completion at week 12. Subjects |
| | contacted the research center if they cleared in between visits and their |
| | final visit occurred 4 weeks after clearing (IGA = 0) had occurred. |
| Statistical Methods: | A t-test were used to analyze the paired data for the presence of TNF-α |
| | and IL-17A in the psoriatic plaques with statistical significance defined |
| | as p less than or equal to 0.5. |

### 2. STUDY VISIT SCHEDULE

| Procedures | Screening | 2 Weeks | 4 Weeks | Week 8 | Week 12 |
|---|---|---|---|---|---|
| Dermatologic Evaluation for | X | | | | |
| Suitability | | | | | |
| Informed consent | X | | | | |
| Inclusion/ | X | | | | |
| Exclusion Criteria | | | | | |
| Concomitant Medications | X | | | | |
| Target Plaque Selection and Body | X | | | | |
| Map | | | | | |
| Investigator Psoriasis Assessment | X | X | X | X | X |
| Subject Psoriasis Plaque | X | X | X | X | X |
| Assessment | | | | | |
| Photography | X | X | X | X | X |
| 10 D-squame tape strips removed | X | X | X | X | X |
| from 2 target plaques (5 strips | | | | | |
| placed in each jar) | | | | | |
| Treatment Product Dispensing | X | | | | |
| Treatment Product Collection | | | If IGA = 0 | If IGA = 0 | |
| Diary Assessment for Compliance | | X | X | X | X |
| Product Accountability and Diary | | | Possible | Possible | X |
| Collection | | | X | X | |

The length of participation for the subjects depended on the presence of an IGA=0 at the treated target plaque. Subjects who were clear at week 4 discontinued treatment and returned for study completion at week 8. Subjects who were clear at week 8 discontinued treatment and returned for study completion at week 12. Subjects who clear in between visits contacted the research center and their final visit occurred 4 weeks after clearing of the target plaque.

### 3. INTRODUCTION

Psoriasis is recognized as a systemic inflammatory disease with skin manifestations of scaling, erythema, and induration. Inflammatory mediators, such as TNF-α and IL-17A, can be down regulated resulting in resolution of the appearance of skin disease through topical, oral, or injectable medications. Monoclonal antibodies to TNF-α and IL-17A, known as injectable biologics, are administered to improve psoriasis, however these same effects can be achieved through topical administration of safe medications for patients with more limited disease. This research examined the effect of a tazarotene/halobetasol lotion on the reduction of TNF-α and IL-17A and how this reduction correlates to psoriasis improvement by noninvasive skin scale sampling and ELISA analysis.

### 4. STUDY OBJECTIVE

The objective was to demonstrate disease modification through anti-inflammatory effects (reduction in TNF-α, IL-17A) of tazarotene/halobetasol lotion in patients with mild to moderate plaque type psoriasis as compared to control.

### 5. STUDY DESIGN OVERVIEW

Male or female subjects with mild to moderate plaque type psoriasis presented to the research center for evaluation for study entry. They were advised to present to the research center with clean skin and no topical products applied to the body, including medications, moisturizers, sunscreens, etc. If found to be suitable by meeting all inclusion criteria and none of the exclusion criteria, subjects underwent selection of 2 target plaques by the dermatologist investigator. Each plaque was graded for erythema, scaling, and induration. An Investigational Global Assessment (IGA) score was assigned for each plaque. Plaques were an IGA of mild (2) or moderate (3) to qualify for inclusion. The subjects also assessed each plaque for redness, thickness, and scaling. Each of the plaques was photographed.

The subjects underwent D-squame tape strip removal of each of the 2 target psoriasis plaques. One plaque served as the tazarotene/halobetasol treatment plaque and the other served as the untreated control. 10 D-squames were obtained with from each plaque. 5 tape strips were placed in each glass jar. One jar contained the even tape strips and the other jar contained the odd tape strips. 10 D-squames were also taken from normal skin to serve as a negative control in an individual without psoriasis. 5 tape strips were placed in each glass jar. One jar contained the even tape strips and the other jar contained the odd tape strips. The D-squames were frozen in a −80° C. freezer for later assessment. Five tape strips were analyzed together to obtain adequate material for LCMS analysis. Two specimens jars were prepared for from each of the 2 target plaques. One jar was analyzed with the second jar serving as a backup. It has been determined from the pilot study that the inflammatory mediator amount is fairly consistent at skin depths reached by 10 tape strips.

The subjects had a number assigned to each of the treatment plaques. The numbers were recorded on a body map. Two copies were prepared. One copy remained at the research center and the second copy was provided to the subject. Each subject received one tube of tazarotene/halobetasol lotion for application to the treated plaque. The second plaque functioned as an untreated control site.

Subjects were provided with a diary. They were instructed to apply the study product once daily at bedtime to the treated plaque only. Following application of the tazarotene/halobetasol lotion to the treated plaque once daily at bedtime, both the treated and untreated plaques received moisturizer application once daily. Care was taken to apply the proper product to the proper plaque. Subjects were instructed to returned to the research center in 2 weeks. A reminder text for compliance was provided prior to the week 2 visit.

Subjects returned to the research center at week 2. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of 10 tape strips for each of the 2 target psoriasis plaques. Five tape strips were placed in each jar with one jar receiving the even tape strips and the other jar receiving the odd tape strips. The D-squames were frozen in a −80° C. freezer for later assessment. Subjects were asked to return to the research center at week 4 or sooner if the treated psoriasis plaque has cleared (IGA-0). If the treated plaque has cleared, subjects contacted the research center and 4 weeks after treatment plaque clearing underwent a final tape stripping procedure and completed their study participation. A reminder text for compliance was sent prior to the week 4 visit.

Subjects returned to the research center at week 4. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal 10 times for each of the 2 target psoriasis plaques. Five tape strips were placed in each jar with one jar containing the even tape strips and the other jar containing the odd tape strips. The D-squames were frozen in a −80° C. freezer for later assessment. Subjects who experienced clearing (IGA-0) of the treatment plaque discontinued treatment and returned to the research at week 8. Subjects who had remaining psoriasis at the site of the treatment plaque continued treatment as specified previously and returned to the treatment center at week 8. A reminder text for compliance was sent prior to the week 8 visit.

Subjects returned to the research center at week 8. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of each of the 2 target psoriasis plaques. 10 tape strips were removed from each plaque. Five tape strips were placed in each jar with the even tapes placed in one jar and the odd tapes placed in the second jar. The D-squames were frozen in a −80° C. freezer for later assessment. Subjects who experienced clearing (IGA-0) at week 8 discontinued treatment and returned to the research at week 12. Subjects who did not have an IGA-0 or clearing of the treatment plaque continued study drug application and returned to the research center at week 12.

Subjects returned to the research center at week 12. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of each of the 2 target psoriasis plaques 10 times to procure 10 tape strips. Five tape strips were placed in each jar with the even tape strips placed in one jar and the odd tape strips placed in a second jar. The D-squames were frozen in a −80° C. freezer for later assessment. All subjects completed their study participation.

The length of participation for the subjects depended on the presence of an IGA=0 at the treated target plaque. Subjects who were clear at week 4 discontinued treatment and returned for study completion at week 8. Subjects who were clear at week 8 discontinued treatment and returned for study completion at week 12. If clearing occurs in between visits, the subjects contacted the research center and the date of clearing was noted and treatment discontinued. Subjects returned for the final tape stripping and study completion 4 weeks after clearing had occurred.

### 6. STUDY POPULATION

6.1 Population description

Healthy male or non-pregnant female subjects 18+ years of age with plaque type psoriasis.

### 6.2 Population Size

10 subjects

### 6.3 Inclusion Criteria

The following items represented the inclusion criteria:
1. Males or non-pregnant females 18+ years of age.
2. Plaque type mild to moderate psoriasis suitable for topical treatment.
3. The presence of 2 plaques suitable for tape stripping
4. Subjects must be willing to allow a series of tape pieces to be pressed and removed from 2 target psoriasis plaques.
5. Subjects must be in general good health as determined from a medical history.
6. Subjects must read and sign the informed consent form after the nature of the study has been fully explained.

### 6.4 Exclusion Criteria

The following items represented the exclusion criteria:
1. Subjects with known allergies or sensitivities to ingredients contained in the test products.
2. Subjects with an allergy to latex or adhesives.
3. Subjects with pustular or erythrodermic psoriasis.
4. Subjects who are pregnant or nursing or planning to become pregnant during the course of the study.
5. Subjects who are currently participating in any other clinical study (i.e., dermal patch, use tests, investigational drug or devices, etc.).
6. Subjects viewed by the investigator as not being able to complete the study.
7. Subjects using any type of lotion, medication, or other topical product to the psoriasis plaques.

### 6.5 Concomitant Medications

All oral and topical prescription medications remained unchanged during the study, but there were no prohibited medications. No topical medications of any kind can were used on the psoriasis plaques, other than that provided.

### 7. CONDUCT OF STUDY: METHODS AND PROCEDURES

### 7.1 Enrollment

### 7.1.1 Informed Consent

A signed informed consent form was obtained from each subject prior to performing any study procedures. No study related procedures or activities were performed until each subject was fully informed and the consent form was signed and dated.

### 7.1.2 Medical History

An abbreviated medical history including current medications was recorded.

### 7.1.3 Dermatological Examination

A dermatological examination was performed.

### 7.1.4 Study Procedures

The subjects were screened for the inclusion and exclusion criteria prior to study enrollment. Only subjects who meet the requirements, have signed an informed consent, and have given a medical history were entered into the study. All other subjects were considered screening failures.

### 7.1.5 Study Material Administration

Subjects will use Duobrii product as currently marketed once daily at bedtime to the assigned psoriasis target plaque and nothing to a control plaque.

### 7.1.6 Screening Procedures

Potential volunteers were enrolled based on the presence of two psoriasis plaques suitable to evaluation and their ability to meet the inclusion/exclusion criteria required for study enrollment.

### 7.2 Study Conduct Procedures

### 7.2.1 Baseline

Male or female subjects with mild to moderate plaque type psoriasis presented to the research center for evaluation for study entry. They were advised to present to the research center with clean skin and no topical products applied to the body, including medications, moisturizers, sunscreens, etc. If found to be suitable by meeting all inclusion criteria and none of the exclusion criteria, subjects underwent selection of 2 target plaques by the dermatologist investigator. Each plaque was graded for erythema, scaling, and induration. An Investigational Global Assessment (IGA) score was assigned for each plaque. Plaques were an IGA of mild (2) or moderate (3) to qualify for inclusion. The subjects also assessed each plaque for redness, thickness, and scaling. Each of the plaques was photographed.

The subjects underwent D-squame tape strip removal of each of the 2 target psoriasis plaques. One plaque served as the tazarotene/halobetasol treatment plaque and the other served as the untreated control. 10 D-squames were obtained with from each plaque. 5 tape strips were placed in each glass jar. One jar contained the even tape strips and the other jar contained the odd tape strips. 10 D-squames were also taken from normal skin to serve as a negative control in an individual without psoriasis. 5 tape strips were placed in each glass jar. One jar contained the even tape strips and the other jar contained the odd tape strips. The D-squames were frozen in a −80° C. freezer for later assessment. Five tape strips were analyzed together to obtain adequate material for LCMS analysis. Two specimens jars were prepared for from each of the 2 target plaques. One jar was analyzed with the second jar serving as a back up. It has been determined from the pilot study that the inflammatory mediator amount is fairly consistent at skin depths reached by 10 tape strips.

The subjects had a number assigned to each of the treatment plaques. The numbers were recorded on a body map. Two copies were prepared. One copy remained at the research center and the second copy was provided to the subject. Each subject received one tube of tazarotene/halobetasol lotion for application to the treated plaque. The second plaque functioned as an untreated control site.

Subjects were provided with a diary. They were instructed to apply the study product once daily at bedtime to the treated plaque only. Following application of the tazarotene/halobetasol lotion to the treated plaque once daily at bedtime, both the treated and untreated plaques received moisturizer application once daily. Care was taken to apply the proper product to the proper plaque. Subjects were instructed to returned to the research center in 2 weeks. A reminder text for compliance was provided prior to the week 2 visit.

### 7.2.2 Week 2

Subjects returned to the research center at week 2. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of 10 tape strips for each of the 2 target psoriasis plaques. Five tape strips were placed in each jar with one jar receiving the even tape strips and the other jar receiving the odd tape strips. The D-squames were frozen in a −80° C. freezer for later assessment. Subjects were asked to return to the research center at week 4 or sooner if the treated psoriasis plaque has cleared (IGA=0). If the treated plaque has cleared, subjects contacted the research center and 4 weeks after treatment plaque clearing underwent a final tape stripping procedure and completed their study participation. A reminder text for compliance was sent prior to the week 4 visit.

### 7.2.3 Week 4

Subjects returned to the research center at week 4. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal 10 times for each of the 2 target psoriasis plaques. Five tape strips were placed in each jar with one jar containing the even tape strips and the other jar containing the odd tape strips. The D-squames were frozen in a −80° C. freezer for later assessment.

Subjects who experienced clearing (IGA=0) of the treatment plaque discontinued treatment and returned to the research at week 8. Subjects who had remaining psoriasis at the site of the treatment plaque continued treatment as specified previously and returned to the treatment center at week 8. A reminder text for compliance was sent prior to the week 8 visit.

### 7.2.4 Week 8

Subjects returned to the research center at week 8. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of each of the 2 target psoriasis plaques. 10 tape strips were removed from each plaque. Five tape strips were placed in each jar with the even tapes placed in one jar and the odd tapes placed in the second jar. The D-squames were frozen in a −80° C. freezer for later assessment. Subjects who experienced clearing (IGA-0) at week 8 discontinued treatment and returned to the research at week 12. Subjects who did not have an IGA=0 or clearing of the treatment plaque continued study drug application and returned to the research center at week 12.

### 7.2.5 Week 12

Subjects returned to the research center at week 12. The dermatologist investigator evaluated each plaque for erythema, scaling, and induration. An Investigational Global Assessment (IGA) score was assigned for each plaque. The subjects also assessed each plaque for redness, thickness, and scaling. Each of the plaques was photographed. The subjects underwent D-squame tape strip removal of each of the 2 target psoriasis plaques 10 times to procure 10 tape strips. Five tape strips were placed in each jar with the even tape strips placed in one jar and the odd tape strips placed in a second jar. The D-squames were frozen in a −80° C. freezer for later assessment. All subjects completed their study participation.

The length of participation for the subjects depended on the presence of an IGA=0 at the treated target plaque. Subjects who were clear at week 4 discontinued treatment and returned for study completion at week 8. Subjects who were clear at week 8 discontinued treatment and returned for study completion at week 12. If clearing occurs in between visits, the subjects contacted the research center and the date of clearing was noted and treatment discontinued. Subjects returned for the final tape stripping and study completion 4 weeks after clearing had occurred.

### 7.3 Sample Analysis

### 7.3.1 Sample Preparation and Analysis

Tape strips were previous collected from the study subjects and were stored at −80° C. until analyzed. The tape strips were allowed to thaw to room temperature (20° C.). To each tape strip, 2 mL of a 1% Triton X-100 distilled water solution was added in a 20 mL sample vial. The samples were agitated for 1 hours at room temperature and inverted by hand to insure consistent mixing. 1 mL of the extraction solution was then clarified by centrifugation at 10,000×g for 2 minutes. 50 μL or 100 μL of the clarified solution was immediately analyzed by ELISA to quantitate the amount of IL-17A or TNF-α, respectively, according to the manufacturer protocols. For IL-17A, samples were diluted 2-fold with sample diluent whereas TNF-alpha samples were analyzed without further dilution. All samples were added to the appropriate ELISA tray within 1.5 hours of preparation.

### 7.3.2 Analytical Equipment

All samples were analyzed with a VarioSkan Lux UV plate reader (ThermoFisher Scientific; Waltham, MA). Data analysis was exported from SkanIt v6.1 (ThermoFisher Scientific; Waltham, MA) for analysis with Excel (Microsoft Corporation). Standard analytical glassware and equipment were used for preparation of the ELISA buffers according to the manufacturer protocols and extraction solutions for recovery of inflammatory mediators from the collected tape strips.

7.3.3 UV plate reader PARAMETERS

All analyses were conducted with UV-vis absorbance at 450 nm.

### 7.3.4 ELISA Kits

ELISA kits for TNF-α and IL-17A were purchased from ThermoFisher Scientific (Waltham, MA).

### 7.3.5 Solvents

All solvents were purchased from commercial sources and used without further purification. All water used in this study was purchased as molecular biological grade and was certified DNAse-free, RNAse-free, and protease-free and used without further purification. Solutions were prepared immediately prior to use and were not stored for longer than 24 hrs to prevent biological growth.

### 7.3.6 Additives

Triton X-100 was purchased from commercial sources and used without further purification.

### 8. SUBJECT COMPLIANCE

Compliance was determined from the diary sheets. Subjects recorded product application and any comments on the provided weekly diary. Diary sheets remained at the study center as part of the source documentation records. No compliance issues were identified and all collected data was used in the final analysis.

### 9. FINAL SUBJECT STATUS

A study termination form was completed for each study subject who received study product. This included subjects who completed the study or who withdrew or were withdrawn from study. 10/10 subjects successfully completed the study. The summary demographic log is presented below.

| | Number of Subjects | Population Percent |
|---|---|---|
| SEX | | |
| Male | 5 | 50 |
| Female | 5 | 50 |
| Ethnicity | | |
| Hispanic | 2 | |
| Non-Hispanic White | 8 | |
| RACE | | |
| Asian | | |
| African American | 1 | 10 |
| Caucasian | 9 | 90 |
| Multiracial | | |
| FITZPATRICK SKIN | | |
| TYPE | | |
| I | 1 | 10 |
| II | 6 | 60 |
| III | 1 | 10 |
| IV | 1 | 10 |
| V | 1 | 10 |
| VI | 0 | 0 |

### 10. STUDY PRODUCTS & ADMINISTRATION

### 10.1 Formulations

The subjects used Duobrii product as currently marketed.

### 10.2 Precautions

Study products should be used in their intended fashion and not orally consumed or placed in the eyes. Please refer to the attached package insert for further instructions.

### 10.3 Study Product Administration

The subjects applied the Duobrii product as instructed to one target plaque once daily at bedtime. These applications continued until the plaque cleared or the study ended, which ever occurred first.

### 10.4 Packaging, Labeling, Distribution

Study products were dispensed in the packaging provided by the sponsor.

### 10.5 Storage and Accountability of Study Product

The study product was stored at room temperature in a locked, limited access area at the study site. Access to the study product was limited to the investigator and staff members designated to dispense study medication. A study product log were used to record the dispensation and returned of all study product. The subject number/initials, and the initials and date of the person dispensing and receiving the returned study product were documented on this form.

### 10.6 Code Disclosure

All subjects received the active study product and a code was maintained as to which target plaque was untreated and which target plaque received the Duobrii product. This information was marked on a body map with one copy remaining at the research center and one copy provided to the subject for reference.

### 11. ADVERSE EVENTS

No adverse reactions or adverse experiences occurred during the conduct of the study.

### 12. STATISTICAL METHODS

A t-test was used to analyze the paired data for the presence of TNF-α and IL-17A in the psoriatic plaques with statistical significance defined as p less than or equal to 0.5.

### 12.1 Measures

### Investigator Assessments:

Plaque Assessments: The dermatologist investigator assessed erythema, induration, and scaling at baseline, week 2, week 4, week 8, and 12. Each of the 2 target plaques was assessed separately.
Plaque IGA Assessments: The dermatologist investigator assessed plaque IGA at baseline, week 2, week 4, week 8, and week 12. Each of the 2 target plaques was assessed separately.

### Subject Assessments:

The subjects assessed plaque redness, thickness, and scaling at baseline, week 2, week 4, week 8, and week 12. Each of the 2 target plaques was assessed separately.

### ELISA:

10 D-squames were removed from each of the 2 target plaques at baseline, week 2, week 4, week 8, and week 12. Five D-squames were placed in each jar with the even tape strips placed in one jar and the odd tape strips placed in a second jar. One jar was analyzed and the other jar served as a backup. Five tape strips provided enough material for analysis. The tape strips were solvent treated to remove the skin scale. The mixture was clarified with centrifugation and the supernatant were analyzed with ELISA to determine the amount of TNF-α and IL-17A. This was repeated for the specimens from each of the 2 target plaques (tazarotene/halobetasol lotion and untreated). The amount of TNF-α and IL-17A were compared for the 2 target plaques at each collection time point (tazarotene/halobetasol lotion and untreated).

### Photography:

High quality jpeg images were taken of all target plaques at baseline, week 2, week 4, week 8, and week 12. These images have been provided to the sponsor.
Note: The length of participation for the subjects depended on the presence of an IGA=0 at the treated target plaque. Subjects who were clear at week 4 discontinued treatment and returned for study completion at week 8. Subjects who were clear at week 8 discontinued treatment and returned for study completion at week 12. Subjects contacted the research center if they cleared in between visits and their final visit occurred 4 weeks after clearing (IGA=0) had occurred.

### 12.2 Sample Size Rationale

A sample size of 10 subjects was chosen by the study sponsor.

### 12.3 Significance Level

Significance was defined at the p<0.05 level based on a two-sided test.

### 13. RESULTS

The results are presented in the attached data tables:
Table 6: Investigator IGA
Table 7: Investigator Target Lesion Assessment
Table 8: Subject Target Lesion Assessment
Table 9: TNF alpha ELISA Assessment
Table 10: IL-17a ELISA Assessment

### 14. DISCUSSION

The results are discussed separately for each data set.

TABLE 6
Investigator IGA
The dermatologist investigator assessed
plaque IGA at baseline, week 2, week 4, week 8, and week
12. Each of the 2 target plaques was assessed separately.
The summary data table, Table 6, is presented below.

| Mann- | | | | | |
|---|---|---|---|---|---|
| Whitney | | | | | |
| Two | | | | | |
| Tailed | | | | | |
| Paired | Baseline | Week 2 | Week 4 | Week 8 | Week 12 |
| Mean T | 2.60 | 1.70 | 1.40 | 1.00 | 0.80 |
| Mean U | 2.60 | 2.70 | 2.70 | 2.70 | 2.50 |
| T v. U p= | 1.000 | 0.004 | <0.001 | <0.001 | <0.001 |

TABLE 7
Investigator Target Lesion Assessment
The dermatologist investigator assessed plaque erythema,
induration, and scaling at baseline, week 2, week 4, week 8, and
week 12. Each of the 2 target plaques was assessed separately.
The summary data table, Table 7, is presented below.
Investigator Target

Mann-

Whitney

| two | Baseline | | | Week 2 | | | Week 4 | | | Week 8 | | | Week 12 | | |
|---|---|---|---|---|---|---|---|---|---|---|---|---|---|---|---|
| tailed | Erythe- | Indur- | Scal- | Erythe- | Indur- | Scal- | Erythe- | Indur- | Scal- | Erythe- | Indur- | | Erythe- | Indur- | |
| paired | ma | ation | ing | ma | ation | ing | ma | ation | ing | ma | ation | Scaling | ma | ation | Scaling |
| Mean T | 2.50 | 2.50 | 2.50 | 1.60 | 1.20 | 1.60 | 1.30 | 0.80 | 1.10 | 1.00 | 0.80 | 0.90 | 1.00 | 0.90 | 0.90 |
| Mean U | 2.50 | 2.50 | 2.50 | 2.50 | 2.70 | 2.70 | 2.70 | 2.70 | 2.70 | 2.70 | 2.70 | 2.70 | 2.60 | 2.60 | 2.60 |
| T v. U p= | 1.000 | 1.000 | 1.000 | 0.017 | 0.002 | 0.003 | <.001 | <.001 | <.001 | <.001 | <.001 | <.001 | <.001 | <.001 | <.001 |

No difference was seen between the treated and untreated plaques at baseline. By week 2, there was a statistically significant improvement in erythema (p=0.017), induration (p=0.002), and scaling (p=0.003) in the treated plaque as compared to the untreated plaque. This improvement continued to be significant (p<0.001) in all parameters from week 4 though week 12 indicating high efficacy for Duobrii product.

TABLE 8
Subject Target Lesion Assessment
The subjects assessed plaque redness, thickness, and
scaling at baseline, week 2, week 4, week 8, and week 12.
Each of the 2 target plaques was assessed separately.
The summary table, Table 3, is presented below.
Subject Target

Mann-

Whitney

| two | Baseline | | | Week 2 | | | Week 4 | | | Week 8 | | | Week 12 | | |
|---|---|---|---|---|---|---|---|---|---|---|---|---|---|---|---|
| tailed | | Thick- | | | Thick- | | Red- | Thick- | | Red- | Thick- | | Red- | Thick- | |
| paired | Redness | ness | Scaling | Redness | ness | Scaling | ness | ness | Scaling | ness | ness | Scaling | ness | ness | Scaling |
| Mean T | 3.10 | 3.20 | 3.20 | 2.20 | 2.10 | 2.10 | 1.80 | 1.80 | 1.80 | 1.10 | 1.10 | 1.10 | 1.00 | 1.00 | 1.00 |
| Mean U | 3.10 | 3.20 | 3.20 | 3.20 | 3.30 | 3.30 | 3.00 | 3.10 | 3.10 | 2.80 | 2.80 | 2.80 | 2.90 | 2.90 | 3.00 |
| T v. U p= | 1.000 | 1.000 | 1.000 | <.001 | <.001 | <.001 | <.001 | <.001 | <.001 | <.001 | <.001 | <.001 | <.001 | <.001 | <.001 |

The subjects rated statistically significant (p<0.001) improvement in the treated

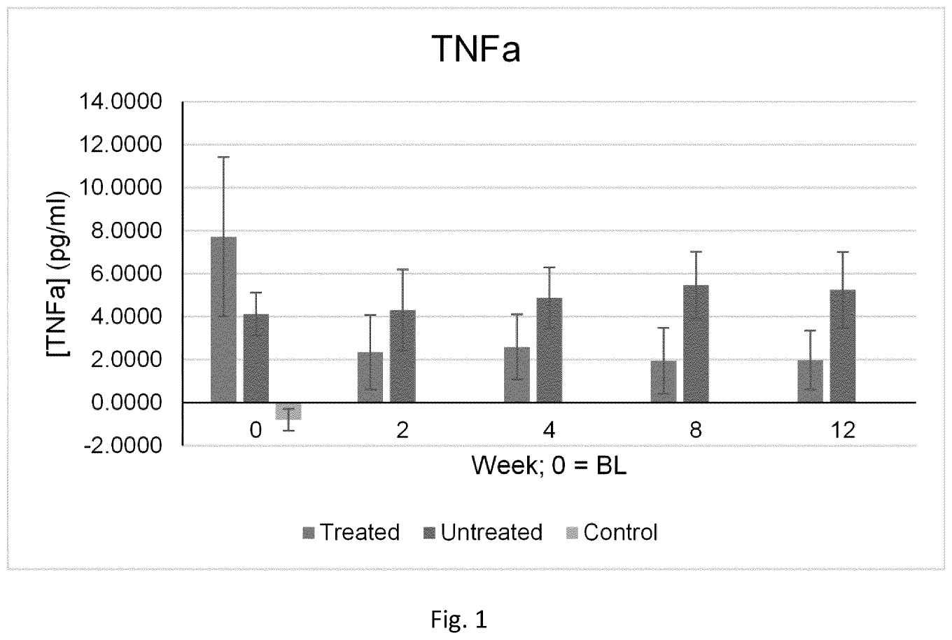

TABLE 9
TNF-α ELISA Assessment
The excellent improvement in the
visual investigator and subject psoriasis assessments was
collaborated by the TNF-α ELISA findings. The summary
graph is shown in FIG. 1. It can be seen from FIG. 1 that
the treated plaque contained less TNF-α at weeks 2, 4,
8, and 12 than the untreated plaque. Thus, even though Duobrii
product is not a biologic, it produced an excellent reduction
in TNF-α. In normal control skin, TNF-α was not detected
above of the limit of quantitation for this assay (0.24 pg/mL).
The t-test analysis summary and p values are presented below in Table 9.

| Time Point | Treated | Untreated | Control | P Value |
|---|---|---|---|---|
| Baseline | 7.72 | 4.11 | −0.80 | 0.053 |
| 2 | 2.34 | 4.31 | | 0.127 |
| 4 | 2.58 | 4.86 | | 0.009 |
| 8 | 1.94 | 5.46 | | 0.006 |
| 12 | 1.97 | 5.24 | | 0.003 |

There was an almost statistically significant (p=0.053) difference between the treated and untreated samples at baseline, however, the level of TNF-α was higher in the treated group, which does not bias the study. No statistically significant difference between the treated and untreated plaques was seen at week 2 (p=0.127). There was statistically less TNF-α at weeks 4, 8, and 12 demonstrating the ability of Duobrii product to decrease TNF-α levels in the psoriatic plaque.

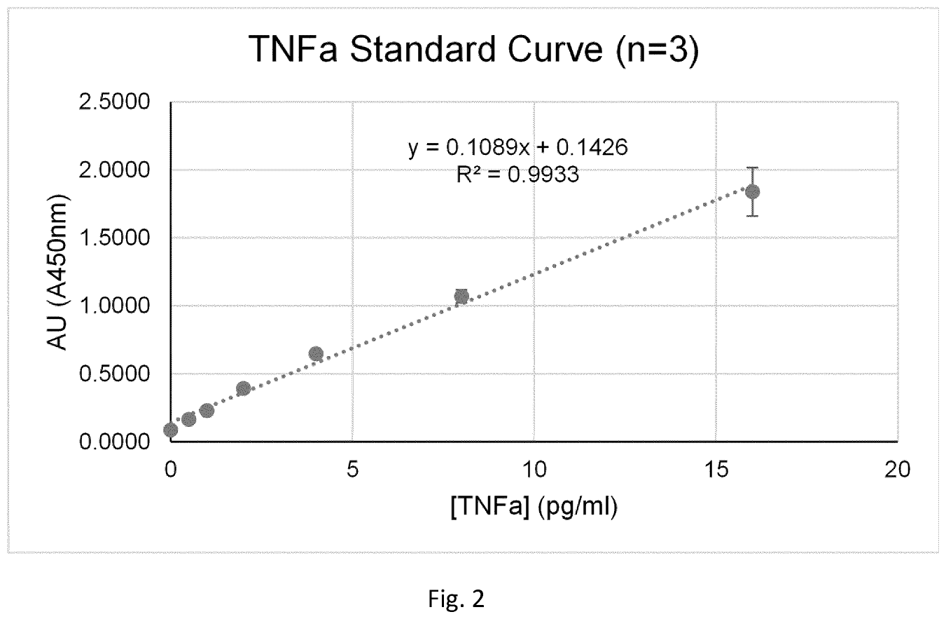

The standard curve used for calibration is presented in FIG. 2. It nicely approximates a straight line (R=0.9933), thus supporting the accuracy of the data analysis.

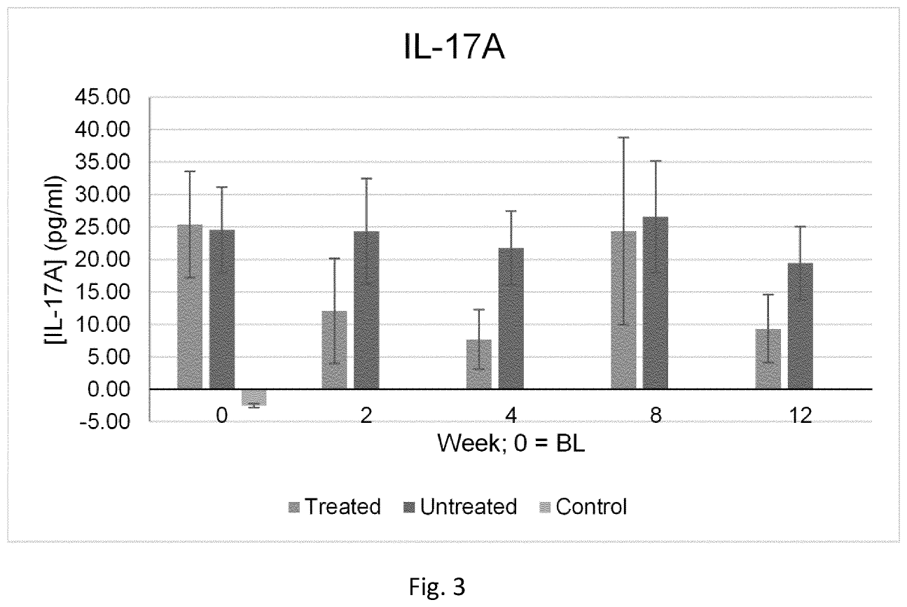

TABLE 10
IL-17A ELISA Assessment
Excellent results were also found for the
IL-17A level reduction in the psoriatic plaque. The results are shown
in FIG. 3. This ELISA assay was repeated twice with a lower sensitivity
ELISA tray used for the second analysis due to unexpectedly large
amounts of IL-17A recovered from the skin scale. The results presented
above represent the lower sensitivity tray. In normal control skin,
IL-17A was not detected above of the limit of quantitation for this
assay (3.2 pg/mL). The summary table, Table 10, for the values obtained
from the t-test analysis are presented below.

| Week | Treated | Untreated | Control | P Value |
|---|---|---|---|---|
| Baseline | 25.38 | 24.57 | −2.52 | 0.897 |
| 2 | 12.08 | 24.31 | | 0.025 |
| 4 | 7.70 | 21.75 | | 0.009 |
| 8 | 24.37 | 26.58 | | 0.666 |
| 12 | 9.35 | 19.42 | | 0.056 |

There was no statistically significant difference between the treated and untreated plaques in their IL-17A levels at baseline. By week 2, there was a statistically significant decrease in the treated plaque IL-17A levels (p=0.024). This statistical difference with reduced IL-17A in the treated plaque continued in week 4 (p=0.009). At week 8, there were some unusual readings of very high IL-17A levels in subject 8 that skewed the data such that statistical significance was not obtained. The data was almost significant at week 12, but again was skewed by subject 8. It is unclear what happened with subject 8. The improvement in IL-17A would have been significant at weeks 2, 4, and 8 but not significant at week 12 if the outlier data from subject 8 were removed from the analysis.

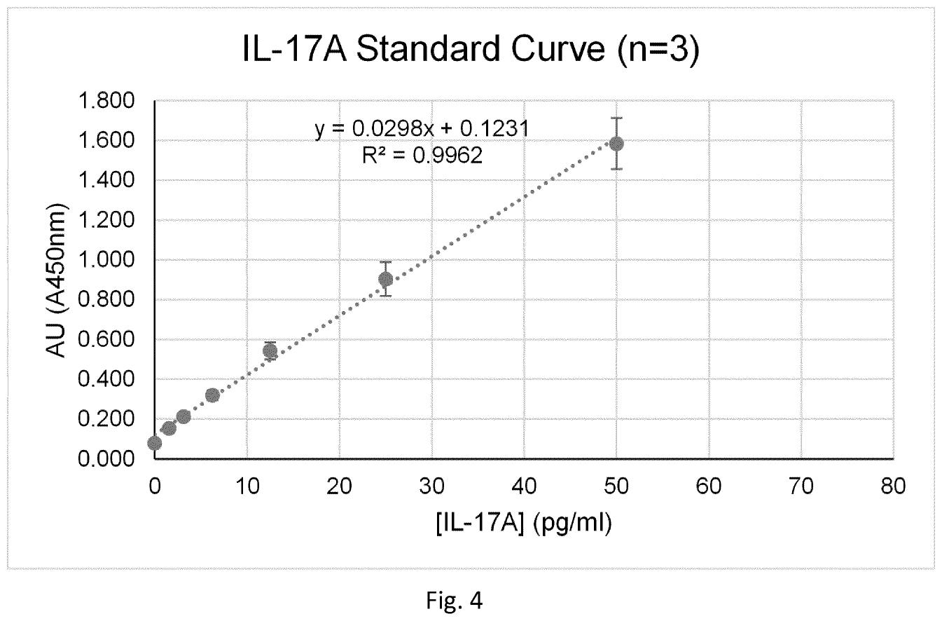

The standard curve for calibration is presented in FIG. 4 and again shows the results are valid (R=0.9962).

### 15. SUMMARY

The results nicely present concurrence between visual psoriasis improvement as assessed by the dermatologist investigator and the subjects. The improvement is captured pictorially for each time point with photographs for both the untreated and treated target plaque. Finally, the improvement with Duobrii product application has been demonstrated in terms of a statistically significant reduction in the inflammatory markers TNF-α and IL-17A characteristic of psoriasis. This research nicely links the clinical findings with a reduction in inflammatory mediators produced by a topical combination lotion containing tazarotene and halobetasol.

### 15.1 Safety Assessment

Incidence of all adverse events reported during the study was summarized. There were no adverse events and the safety assessment for the study product was excellent.

### 15.2 Endpoint Assessment

### 15.2.1 Primary Efficacy Endpoint

The primary efficacy endpoint was the decrease in TNF-α and/or IL-17A after achieving an IGA of 0 with tazarotene/halobetasol lotion as compared to control. The primary efficacy endpoint was met, however, not all subjects achieved an IGA of 0.

### 15.2.1 Secondary Efficacy Endpoint

The secondary efficacy endpoint was the decrease or maintained decrease in TNF-α and/or IL-17A after treatment for 4 weeks with tazarotene/halobetasol lotion as compared to the no treatment control. The secondary efficacy endpoint was met.

What is claimed is:

1. A method of treating a skin disease or condition associated with elevated levels of IL-17 in a subject in need thereof, the method comprising applying to an affected area on the body of the subject a composition comprising:
(a) a corticosteroid or a pharmaceutically acceptable salt or ester thereof, at a positive concentration of less than 0.05 percent by weight of the composition;
(b) a retinoid or a pharmaceutically acceptable salt or ester thereof at a positive concentration of less than 0.09 percent by weight of the composition; and
(c) a dermatologically acceptable excipient.

2. The method of claim 1, wherein the corticosteroid or a pharmaceutically acceptable salt or ester thereof is selected from the group consisting of halobetasol, clobetasol, betamethasone, diflorasone, and fluocinonide.

3. The method of claim 1, wherein the corticosteroid or a pharmaceutically acceptable salt or ester thereof is selected from the group consisting of halobetasol propionate, clobetasol propionate, betamethasone dipropionate, diflorasone diacetate, and fluocinonide.

4. The method of claim 1, wherein the retinoid is selected from the group consisting of tazarotene, bexarotene, and adapalene.

5. The method of claim 1, wherein the corticosteroid is halobetasol propionate, and the retinoid is tazarotene.

6. The method of claim 1, wherein the dermatologically acceptable excipient comprises a liquid oil component and an aqueous component.

7. The method of claim 6, wherein the liquid oil component comprises one or more materials that are practically insoluble or insoluble in water and which are liquid at room temperature of 22° C.

8. The method of claim 6, wherein the liquid oil component comprises a dicarboxylic acid ester, a monocarboxylic ester, or a combination thereof.

9. The method of claim 8, wherein the liquid oil component comprises a dicarboxylic acid ester.

10. The method of claim 8, wherein the liquid oil component comprises a monocarboxylic acid ester.

11. The method of claim 8, wherein the liquid oil component further comprises mineral oil or light mineral oil.

12. The method of claim 11, wherein the liquid oil component further comprises light mineral oil.

13. The method of claim 6, wherein the composition further comprises one or more humectants, preservatives, chelating agents, emulsifying agents, and/or thickening agents.

14. The method of claim 13, wherein the composition further comprises an emulsifying agent.

15. The method of claim 6, wherein the composition further comprises a pH adjusting agent.

16. The method of claim 1, wherein the skin disease or condition is an inflammatory, autoimmune, or oncological skin disease or condition.

17. The method of claim 16, wherein the inflammatory skin disease or condition is selected from the group consisting of atopic dermatitis, pustular psoriasis, acne keloidalis nuchae (ACN), psoriatic arthritis, dermatitis herpetiformis (DH), bullous pemphigoid (BP), pemphigus vulgaris (PV), ankylosing spondylitis, pityriasis rubra pilaris (PRP), and hidradenitis suppurative.

18. The method of claim 16, wherein the autoimmune skin disease or condition is selected from the group consisting of vitiligo and alopecia areata.

19. The method of claim 16, wherein the oncological skin disease or condition is selected from the group consisting of non-melanoma skin cancer (NMSC), melanoma, cutaneous T-cell lymphoma (CTCL).

20. The method of claim 1, wherein the skin disease or condition is cutaneous T-cell lymphoma (CTCL).

21. The method of claim 1, wherein the skin disease or condition is acne keloidalis nuchae (ACN).

22. The method of claim 1, wherein the skin disease or condition is pustular psoriasis.

23. The method of claim 6, wherein the composition is an oil-in-water emulsion.

24. The method of claim 23, wherein the composition is a lotion.

25. The method of claim 23, wherein the composition is a cream.

26. A method of treating a skin disease or condition associated with elevated levels of IL-17 in a subject in need thereof, the method comprising applying to an affected area on the body of the subject a pharmaceutical composition comprising:
halobetasol propionate at a concentration of 0.01 percent by weight of the composition;
tazarotene at a concentration of 0.045 percent by weight of the composition;
a liquid oil component, wherein the liquid oil component comprises one or more materials that are practically insoluble or insoluble in water and which are liquid at room temperature of 22° C.; and
an aqueous component;
wherein the pharmaceutical composition is formulated as a lotion.

27. The method of claim 26, wherein the halobetasol propionate is dissolved in the liquid oil component at room temperature.

28. The method of claim 26, wherein the tazarotene is dissolved in the liquid oil component at room temperature.

29. The method of claim 26, wherein the liquid oil component comprises a dicarboxylic acid ester, a monocarboxylic ester, or a combination thereof.

30. The method of claim 29, wherein the liquid oil component comprises a dicarboxylic acid ester.

31. The method of claim 29, wherein the liquid oil component comprises a monocarboxylic acid ester.

32. The method of claim 29, wherein the liquid oil component further comprises mineral oil or light mineral oil.

33. The method of claim 32, wherein the liquid oil component further comprises light mineral oil.

34. The method of claim 26, wherein the composition further comprises one or more humectants, preservatives, chelating agents, emulsifying agents, and/or thickening agents.

35. The method of claim 34, wherein the composition further comprises an emulsifying agent.

36. The method of claim 26, wherein the composition further comprises a pH adjusting agent.

37. The method of claim 26, wherein the skin disease or condition is an inflammatory, autoimmune, or oncological skin disease or condition.

38. The method of claim 37, wherein the inflammatory skin disease or condition is selected from the group consisting of atopic dermatitis, pustular psoriasis, acne keloidalis nuchae (ACN), psoriatic arthritis, dermatitis herpetiformis (DH), bullous pemphigoid (BP), pemphigus vulgaris (PV), ankylosing spondylitis, pityriasis rubra pilaris (PRP), and hidradenitis suppurative.

39. The method of claim 37, wherein the autoimmune skin disease or condition is selected from the group consisting of vitiligo and alopecia areata.

40. The method of claim 37, wherein the oncological skin disease or condition is selected from the group consisting of non-melanoma skin cancer (NMSC), melanoma, cutaneous T-cell lymphoma (CTCL).

41. The method of claim 26, wherein the skin disease or condition is cutaneous T-cell lymphoma (CTCL).

42. The method of claim 26, wherein the skin disease or condition is acne keloidalis nuchae (ACN).

43. The method of claim 26, wherein said applying is carried out once daily for longer than two weeks.

44. The method of claim 26, wherein said applying is carried out once daily for twelve weeks.

45. The method of claim 43, wherein the method continues to provide relief for a period of four weeks after eight weeks of applying the composition to the affected area on the body of the subject.

46. A method of inhibiting interleukin-17 activity in a subject in need thereof, the method comprising administering to the subject a composition comprising:
(a) a corticosteroid or a pharmaceutically acceptable salt or ester thereof, at a positive concentration of less than 0.05 percent by weight of the composition, wherein the corticosteroid is selected from the group consisting of halobetasol, clobetasol, betamethasone, diflorasone, and fluocinonide;
(b) a retinoid or a pharmaceutically acceptable salt or ester thereof at a positive concentration of less than 0.09 percent by weight of the composition; and
(c) a dermatologically acceptable excipient.

47. A method of treating a patient suffering from a skin disease or condition associated with elevated levels of IL-17, the method comprising: (a) determining whether the patient has an elevated level of IL-17; and (b) if the patient has an elevated level of IL-17, administering to the patient an effective amount of a composition comprising or susceptible to a medical condition that is mediated directly or indirectly by IL-17, the method comprising administering to the patient a composition for a time sufficient to treat the disease or condition, wherein the composition comprises:
(a) a corticosteroid or a pharmaceutically acceptable salt or ester thereof, at a positive concentration of less than 0.05 percent by weight of the composition, wherein the corticosteroid is selected from the group consisting of halobetasol, clobetasol, betamethasone, diflorasone, and fluocinonide;
(b) a retinoid or a pharmaceutically acceptable salt or ester thereof at a positive concentration of less than 0.09 percent by weight of the composition; and
(c) a dermatologically acceptable excipient.
